(19) United States
(12) Patent Application Publication
(10) Pub. No.: US 2023/0131219 A1
(43) Pub. Date: Apr. 27, 2023

(54) USE OF AGONISTS TO AUGMENT CAR T FUNCTION IN SOLID TUMORS

(71) Applicants: The University of North Carolina at Chapel Hill, Chapel Hill, NC (US); The United States of America, as Represented by the Secretary, Department of Health and Human Servic, Bethesda, MD (US)

(72) Inventors: Jonathan Serody, Chapel Hill, NC (US); Nicholas P. Restifo, Chevy Chase, MD (US); Nuo Xu, Chapel Hill, NC (US)

(21) Appl. No.: 17/775,262

(22) PCT Filed: Nov. 9, 2020
(86) PCT No.: PCT/US2020/059751

§ 371 Date: May 6, 2022

Related U.S. Application Data
(60) Provisional application No. 63/069,649, filed on Aug. 24, 2020.
(60) Provisional application No. 62/932,913, filed on Nov. 8, 2019.

(51) Int. Cl. A61K 35/17; A61K 39/395; A61P 35/00; C12N 5/0783; A61K 38/17; A61K 31/352

(52) U.S. Cl. CPC A61K 35/17; A61K 39/3955; A61P 35/00; C12N 5/0636; A61K 38/1774; A61K 38/177; A61K 31/352; A61K 2039/505

(57) ABSTRACT

Disclosed herein is a chimeric antigen receptor T cell therapy for treating patients having a cancer, such as a cancer having one or more solid tumors.

### RELATED APPLICATION(S)

This application claims the benefit of U.S. Provisional Application No. 63/069,649, filed on Aug. 24, 2020, and U.S. Provisional Application No. 62/932,913, filed on Nov. 8, 2019. The entire teachings of the above applications are incorporated herein by reference.

### GOVERNMENT SUPPORT

This invention was made with government support under Grant Nos. CA058223 and BC010763 awarded by the National Institutes of Health. The government has certain rights in the invention.

### BACKGROUND OF THE INVENTION

Chimeric antigen receptor (CAR) T cells have substantial activity against CD19-expressing B cell malignancies in humans^{1,2}. These receptors fuse the single-chain variable fragment (scFv) of an antibody specific to a tumor target with the signaling molecules of an effector T cell. This provides these cells with the specificity of an antibody and the effector function of T cells^{3,4.}

Treatment of patients with solid tumors using CAR T cell therapy, however, has been less successful^{5,6}. Potential barriers to CAR T cell efficacy against solid tumors include the suboptimal migration and persistence of CAR-T cells in the tumor microenvironment (TME), impaired function mediated by the immunosuppressive TME, and CAR T cell exhaustion. Previous work has shown that Th17 and Tc17 cells have enhanced persistence in solid tumors as compared to conventional Th1 cells^{7,8}. It would be beneficial to determine if CAR T cells generated from Th/Tc17 cells are capable of persisting in the immunosuppressive TME of solid tumors.

### SUMMARY OF THE INVENTION

Although CAR T cells targeting CD19 have been successful in treating patients with B cell malignancies^{1,2}, the clinical activity of CAR T cells in patients with solid tumors has been modest^{5,6}. Using a rational and sequential approach, a combinatorial immunotherapy strategy was implemented based on CAR T cells polarized to a Th/Tc17 phenotype, anti-PD1 mAb, STING agonist DMXAA and anti-GR-1 mAb.

Described herein are improved methods for enhancing CAR T cell trafficking and persistence in combination with methods of altering the immunosuppressive tumor microenvironment (TME) to sustain anti-tumor effects of the CAR T cells in solid tumors.

Described herein are methods of treating a disease. The methods include administering to a subject a chimeric antigen receptor modified T cell (CAR T cell) and a STING agonist.

In some embodiments, the CAR T cell is a second generation CAR T cell or a third generation CAR T cell. In some embodiments, the CAR T cell is a Th/Tc17 CAR T cell or a 7/15 CAR T cell.

In some embodiments, the STING agonist is a viral or bacterial product, such as DMXAA or cGAMP. In some embodiments, the CAR T cell and STING agonist are administered concurrently. In other embodiments, the CAR T cell and STING agonist are administered sequentially.

In some embodiments, the methods described herein further include administering to the subject a myeloid derived suppressor cell (MDSC) depleting agent. In some embodiments, the MDSC depleting agent is an antibody, e.g., an anti-Gr1 antibody. In some embodiments, the MDSC depleting agent is administered concurrently with the CAR T cell and the STING agonist. In other embodiments, the MDSC depleting agent is administered sequentially with the CAR T cell and the STING agonist.

In some embodiments, the methods described herein further include administering to the subject a PD-1 inhibitor. In some embodiments, the PD-1 inhibitor is an anti-PD-1 antibody. In some embodiments, the PD-1 inhibitor is administered concurrently with the CAR T cell and the STING agonist. In other embodiments, the PD-1 inhibitor is administered sequentially with the CAR T cell and the STING agonist. In some embodiments, the methods described herein further include inactivating PD-1 in the CAR T cell using gene editing, e.g., using at least one of CRISPR, TALEN, or ZFN.

In some embodiments, the disease is cancer, e.g., breast cancer. In some embodiments, the disease is selected from the group consisting of a sarcoma, a carcinoma, and a lymphoma. In certain embodiments, the disease is a solid tumor.

Also described herein are methods of treating cancer. The methods administering to a subject a Th17 CAR T cell, a STING agonist, a MDSC depleting agent, and a PD-1 inhibitor.

In some embodiments, the Th17 CAR T cell, STING agonist, MDSC depleting agent, and the PD-1 inhibitor are administered concurrently. In alternative embodiments, the Th17 CAR T cell, STING agonist, MDSC depleting agent, and the PD-1 inhibitor are administered sequentially.

In some embodiments, the STING agonist is DMXAA. In some embodiments, the MDSC depleting agent is an antibody. In some embodiments, the MDSC depleting agent is an anti-Gr-1 antibody. In some embodiments, the PD-1 inhibitor is an anti-PD-1 antibody.

In some embodiments, the cancer is selected from the group consisting of a sarcoma, a carcinoma, and a lymphoma. In some embodiments, the cancer is breast cancer. In some embodiments, the cancer is a solid tumor. In some embodiments, the subject exhibits symptoms of a cytokine-release like syndrome, and further comprising administering to the subject an anti-IL-6 antibody.

Also disclosed herein are methods of producing Th/Tc17 CAR T cells. The methods include transducing CD4 and CD8 activated T cells with LH28z virus; culturing the transduced T cells with Th17 cytokine cocktail; reactivating the cultured T cells; and culturing the reactivated T cells in Th17 cytokine cocktail plus IL-23.

In some embodiments, the Th17 cytokine cocktail comprises TGF-β1, IL-1β, TNF, IL-6, anti-IFNγ, and anti-IL-2. In some embodiments, the T cells are activated using anti-CD3 and anti-CD28. In some embodiments, the cultured T cells are reactivated using anti-CD3 and anti-CD28. In some embodiments, the LH28z virus is prepared from the LH28z construct comprising a scFv, hinge and transmembrane domain from CD8, and intracellular domains from CD28 and CD3ζ.

### BRIEF DESCRIPTION OF THE DRAWINGS

The patent or application file contains at least one drawing executed in color. Copies of this patent or patent application publication with color drawings will be provided by the Office upon request and payment of the necessary fee. FIGS. 1A-1J demonstrate Th/Tc17 CAR-T cells exhibit enhanced early control of tumor growth over 7/15 CAR-Ts due to enhanced persistence in the tumor. FIG. 1A provides a schematic of the LH28z CAR cassette encoding the scFv (7.16.4), hinge and transmembrane domain from CD8, and intracellular domains from CD28 and CD3ζ. FIG. 1B shows expression of CAR-T receptor on transduced murine 7/15 CD4⁺ (left) and CD8⁺ (right) T cells. FIG. 1C provides representative flow cytometry histograms depicting viability of Neu⁺ NT2 tumor cells in vitro after 3 days of co-culture with 7/15 CAR-Ts. Tumor cells were pre-labeled with CFSE and plated prior to the addition of CAR-Ts at a 1:1 ratio with tumor cells. FIG. 1D shows intracellular staining illustrating IFN-γ and TNF production by 7/15 CAR-Ts after co-culture with Neu⁺ cells or Neu⁻ 3T3 cells at a 2:1 ratio. FIG. 1E shows tumor area change (tumor area prior to therapy subtracted from area following therapy) was determined and compared in FVB-neu mice that received 7/15 CAR-Ts or mock transduced T cells (Mock Ts). FIG. 1F shows expression of CAR-T receptor on transduced Th17 (left) or Tc17 (right) cells. FIG. 1G shows in vitro killing of NT2 tumor cells by 7/15 CAR-T and Th/Tc17 CAR-T cells following overnight culture. FIG. 1H provides histogram flow plots of IL-17A, TNF and IFN-7 secretion by Th/Tc17 CAR-T and 7/15 CAR-T compared to mock T cells after co-culture with Neu⁺ cells at 3:1 ratio for 6 hours. FIG. 1I shows tumor area change was calculated and compared in FVB-neu mice that received Th/Tc17 CAR-Ts or 7/15 CAR-Ts. FIG. 1J shows detection of CD4 and CD8 CAR-Ts in spleen or tumor by flow cytometry 5d after injection of 7/15 CAR-Ts or Th/Tc17 CAR-Ts. Data from FIGS. 1B-1D are shown as mean±s.d. (n=3) and FIG. 1E represents 7 mice per group. Data from FIGS. 1F-1H shown as mean±s.d. (n=3). Data from FIG. 1I represent 7-8 mice per group, FIG. 1J represent 5 mice per group. * P<0.05, ** P<0.01, significance was determined by Student's t-test or two-way ANOVA when comparing tumor growth change between groups.

FIGS. 2A-2E demonstrate tumor control by Th/Tc17 CAR-T cells is enhanced by DMXAA injection due to altered composition of tumor infiltrating immune cells. NT2 tumors were orthotopically injected into murine mammary pads. 21 days later 500 μg of DMXAA was injected at a site distal to the tumor site, followed by injection of 3×10⁶ mock-T cells 7/15 CAR-T or Th/Tc17 CAR-T cells (CD4:CD8 at ˜1:1 ratio). FIG. 2A provides representative flow cytometry histograms of CAR-Ts detection at in the TME (left) and summary of accumulation of CAR-T cells (right). FIG. 2B shows change in tumor area was measured over 7 days after treatment. FIG. 2C shows Kaplan-Meier survival curve for the treatment cohorts. End point criteria for sacrifice was a tumor area of 200 mm² or greater. FIG. 2D shows tumor area change 14 days after indicated therapy. FIG. 2E shows t-SNE analysis of pooled single cell sequencing data generated from tumor infiltrating CD45⁺ immune cells 7 days after receiving Th/Tc17 CAR-Ts in the presence or absence of DMXAA. Data were analyzed by unsupervised clustering and populations determined by expression of key markers, including Cd3e (T cells), Adgre1 (macrophage) and Itgam and/or Itgax (myeloid cells). Right panel shows further classification of sub-population between DMXAA treatment (+DMXAA) and non DMXAA treated (−DMXAA) mice. The gating from the flow cytometry histograms was performed using FMO for each individual experiment. FIGS. 2B-2D shows 5-10 mice. FIG. 2E shows 3 mice in each treatment group. * P<0.05, ** P<0.01, *** P<0.001 significance was determined by Student's t-test or two-way ANOVA when comparing tumor growth change between groups.

FIGS. 3A-3J demonstrate DMXAA treatment enhances the number and cytotoxicity of Tc17 CAR-T cells in the TME, though they eventually become exhausted. FIG. 3A shows T cell populations were subdivided based on expression of Cd8 and Cd44 (activated CD8 T cells), Cd44 (naïve T cells), Cd4 and Foxp3 (regulatory T cells, T_{reg}). CD8 T cells were compared between the +DMXAA and −DMXAA treatment groups, and each sub-population was examined further as described below. FIG. 3B provides t-SNE plots of CD8 T cells for Tc17 related genes and FIG. 3C provides t-SNE plots of CD8 T cells for Tc1 related genes. FIG. 3D provides violin plots depicting the distribution and change in expression of genes identified in FIG. 3B and FIG. 3C. FIG. 3E shows validation of single-cell sequencing data using flow cytometry to detect CD8 CAR-T cells. Representative flow plots (left), frequency (middle) and number (right) of CD8 CAR-T cells. FIG. 3F provides an analysis of the correlation between tumor growth change and absolute cell number of CAR-expressing CD8 T cells/cm² tumor. FIG. 3G shows activated or exhausted T cell cluster was selected from unsupervised clustering result of tumor infiltrating immune cells from 7 days and 10 days after Th/Tc17 CAR+DMXAA therapy and is shown as a t-SNE plot. FIG. 3H provides t-SNE plot (left) and Violin plot (right) of T cells for genes that are associated with T cells exhaustion. FIG. 3I provides t-SNE plot (left) and Violin plot (right) of T cells for genes that are associated with T cells effector function. FIG. 3J provides t-SNE plot (left) and Violin plot (right) of T cells for genes that are associated with chemokine secretion. *indicates Q<0.05 in the change of percentage expression cells and fold change is larger than 2. # indicates Q<0.05 in the change of cell number. Q value is determined by gene specific analysis (GSA) from Partek flow workstation. Blue * or # indicates change is significant higher for +DMXAA VS −DMXAA or 7 Days VS 10 Days and Red * or # indicates the change is significant higher for −DMXAA VS +DMXAA or 10 Day VS 7 Days. Single cell sequencing data represent 2-3 mice/treatment group and flow cytometric analysis is pooled from 2 independent experiment for n=8.

FIGS. 4A-4M demonstrate DMXAA reduces the accumulation of suppressive macrophages and enhances the trafficking of T cells in the TME, but this effect is eventually lost due to the return of immunosuppressive cells. FIG. 4A provides t-SNE plot of macrophage populations identified by unsupervised clustering (FIG. 2) were selected and classified as M1- or M2-like and compared between mice receiving therapy with or without DMXAA treatment. FIG. 4B provides a heat map of genes identified in FIG. 4A exhibiting a greater than 2-fold change in expression following DMXAA treatment with a significant difference of P<0.05 (determined by GSA analysis). FIG. 4C provides t-SNE plot (left) and violin plots (right) showing expression of M2-related genes. FIG. 4D provides t-SNE plot (left) and violin plots (right) showing expression of M1-related genes. FIG. 4E shows tumor area prior to and after 7 days treatment with Th/Tc17 CAR-T and DMXAA therapy in the following injection of clodronate or control liposomes (n=5 mice per group). FIG. 4F provides t-SNE plot of myeloid populations identified by unsupervised clustering (FIG. 2) were selected and classified as inflammatory myeloid cells or suppressive myeloid cells and compared between mice in the presence or absence DMXAA treatment. FIG. 4G provides heat map of genes identified in FIG. 4F exhibiting a greater than 2-fold change in expression following DMXAA treatment with a significant difference of P<0.05 (determined by GSA analysis). FIG. 4H provides t-SNE plot depicting genes preferentially expressed in inflammatory myeloid cells. FIG. 4I provides violin plots showing distribution and change in expression of genes highlighted in FIG. 4H. Myeloid cells from unsupervised clustering 7 days and 10 days after therapy were selected. FIG. 4J provides t-SNE plot of myeloid cells between 7 days and 10 days after therapy. FIG. 4K provides heat map of pro-inflammatory or suppressive genes that exhibit a greater than 2-fold change in expression following DMXAA treatment with a significant difference of P<0.05. FIG. 4L provides t-SNE plot of myeloid cells illustrating genes associated with the inflammatory and suppressive functions of myeloid cells. FIG. 4M provides violin plots showing distribution and change of indicated gene expression in FIG. 4L. *indicates P<0.05. Significance was determined by Student's t-test. Single cell sequencing data represent 2-3 mice/treatment group.

FIGS. 5A-5D demonstrate DMXAA is required for tumor remission after Th/Tc17 CAR-T, DMXAA, anti-PD-1 and anti-Gr1 treatment. Animals received injections of anti-PD-1 (200 g/mouse) and anti-Gr1 (300 g/mouse) twice weekly, beginning a day after CAR-T injection in additional to therapy described in FIG. 2 (Th/Tc17 CAR+D+aP+aG). FIG. 5A provides a schematic describing therapy schedule. FIG. 5B shows summary of tumor growth (in area) in the first 3 weeks after CAR-T therapy. FIG. 5C shows change in tumor area was assessed 7 days after administration of Th/Tc CAR+triple therapy or in the absence of DMXAA, anti-PD-1, anti-Gr-1 or CAR-T cells (mock T cells to model the absence of CAR-T cells). FIG. 5D provides Kaplan-Meier survival curve indicating mortality over 100 days for the four treatment groups. Each group includes 9-12 mice pooled from a minimum of two independent experiments. * P<0.05, ** P<0.01, *** P<0.001, **** P<0.0001, significance was determined by Student's t-test or two-way ANOVA when comparing tumor growth change between groups or log-rank Mantel-Cox test when comparing survival.

FIGS. 6A-6G demonstrate depletion of myeloid cells with anti-Gr1 during Th/Tc17 CAR+D+aP regimen leads to cytokine release-like syndrome. Mice received Th/Tc17 CAR+D+P with or without twice weekly anti-Gr-1 injections after CAR-T therapy. FIG. 6A provides quantification of weight loss as percentage of total change in body weight following Th/Tc17 CAR+D+P or Th/Tc17 CAR+D+aP+aG treatment (n=6 mice/group). FIG. 6B provides Kaplan-Meier survival curve indicating mortality over 20 days in the presence or absence of anti-Gr-1 treatment. FIG. 6C shows change in tumor area 7 days after Th/Tc17 CAR+D+P or Th/Tc17 CAR+D+aP+aG treatment in mice that survived but lost more than 10% of their initial weigh (CRS-like) compared to mice that eventually died from CRS-like symptoms (severe CRS-like) (n=4-6 mice/group). FIG. 6D shows serum cytokine levels of CCL2, mG-CSF and IL-6 measured 5 days after therapy (n=4 mice per group). FIG. 6E provides quantification of weight loss as percentage of total change in body weight following Th/Tc17 CAR+D+aP+aG+aIL-6 or Th/Tc17 CAR+D+aP+aG treatment (n=7 mice/group). FIG. 6F provides Kaplan-Meier survival curve indicating mortality over 20 days in the presence or absence of anti-IL-6 mAb treatment. FIG. 6G shows tumor growth change at 14 days after the therapy in the presence or absence of anti-Gr-1 and/or anti-IL-6 mAb. Data are shown as mean±s.d. * P<0.05, ** P<0.01, *** P<0.0001, significance was determined by Student's t-test, Mann-Whitney test when comparing serum cytokine, and log-rank Mantel-Cox test when comparing survival.

FIGS. 7A-7E demonstrate IL-7/15 cultured LH28BBz or LHBBz CAR-T cells fail to control in vitro or in vivo tumor growth control compared to LH28z CAR-T cells. FIG. 7A provides a schematic of the different CAR cassettes encoding the scFv (7.16.4), hinge and transmembrane domain from CD8, and intracellular domains from CD3ζ, 4-1BB and/or CD28. FIG. 7B shows expression of CAR-T receptor on IL-7/15 cultured LH28BBz or LHBBz CAR-T cells compared to LH28z CAR-T cells. FIG. 7C shows ratio of CD4⁺:CD8⁺ CAR T cells after 6 days of expansion with IL-7 and IL-15. FIG. 7D shows NT2 cells were co-cultured with different ratios of indicated CAR-T cells for 6 hours, and tumor cell ATP activity was measured. Tumor ATP level indicating death of tumor cells was characterized at different ratios of mock-T cells, LH28z or LH28BBz CAR-T cells. FIG. 7E shows tumor area change was calculated following the administration of IL-7/15 cultured LHBBz or LH28z CAR-Ts (3×10⁶ CAR-T cells intravenously at 1:1 CD4⁺:CD8⁺ ratio) into tumor bearing mice when the tumor size reached 50 mm². Data in FIG. 7C are shown as mean±s.d. (n=3). Data in FIG. 7D represent 5-8 mice per group and significance was determined by two-way ANOVA.

FIGS. 8A-8E demonstrate 7/15 CAR-Ts fail to control NT2 tumor growth in vivo compared to Th/Tc17 CAR-Ts. FIG. 8A provides a schematic of the tumor model in which 5×10⁴ NT2 tumor cells were injected orthotopically into the mammary fat pad at day −21. Mice received 3×10⁶ CAR-T cells intravenously at 1:1 CD4⁺:CD8⁺ ratio at day 0 when the tumor size reached 50 mm². FIG. 8B shows tumor area change was calculated following injection of 7/15 CAR-T prior to lymphopenia induced by 5Gy total body irradiation. FIG. 8C shows tumor area change was calculated following the administration of anti-PD-1 (200 μg/mouse) twice a week following 7/15 CAR-Ts injection. FIG. 8D provides representative flow cytometry histograms showing detection of CAR-T cells within the tumor 5 days after 7/15 CAR-Ts infusion. FIG. 8E provides representative flow plots showing detection of CAR-T cells within the tumor or spleen 5 days after 7/15 CAR-Ts or Th/Tc17 CAR-Ts infusion. Data are shown as mean±s.d. with 4-8 mice per group.

FIGS. 9A-9B demonstrate DMXAA treatment induces global changes in immune cells of the TME 7 days after therapy. FIG. 9A shows immune cells were analyzed using unsupervised clustering and populations were determined by expression of key markers, including Cd3e (T cells), Itgam+Adgre1+ (macrophage) and Itgam+Adgre1− (myeloid cells). FIG. 9B shows comparison of frequency of indicated cell populations between +DMXAA and −DMXAA treated animals. * P<0.05, significance was determined by Student's t-test. Single cell sequencing data represent 3 mice/treatment group.

FIGS. 10A-10D demonstrate Th/Tc17 CAR-Ts, but not Tc17 CAR-Ts alone, control tumor growth in the presence of DMXAA. Tumor infiltrating T cells were isolated 7 days after therapy and CAR expression was determined by flow cytometry. FIG. 10A shows determination of change in tumor area 7 days after injection of Tc17 or Th/Tc17 CAR-Ts (n=5-8 mice per group). FIG. 10B provides assessment of intratumoral CD8⁺ CAR-T cells was performed 7 days after therapy. FIGS. 10C-10D show frequency and absolute number of CD8⁺ CAR-T cells per mm² tumor 7 days after therapy (n=3-9 mice per group). Data shown as mean±s.d. * P<0.05, ** P<0.01, significance was determined by Student's t-test. Data represent of one of two independent experiments.

FIGS. 11A-11F demonstrate DMXAA treatment enhances the number and cytotoxicity of Th17 CAR-T cells in the TME. FIG. 11A shows CD4⁺ T cells were subdivided based on absence of Cd44 (naïve T cells), expression of Cd4 and Cd44 (activated CD4 T cells), Cd4 and Foxp3 (T_{reg}). CD4⁺ T cells were compared between the +DMXAA and −DMXAA treatment groups, and each sub-population was examined further as described below. FIG. 11B provides t-SNE plot of CD4⁺ T cells for Th17 related genes. FIG. 11C provides t-SNE plot of CD4⁺ T cells for Th17/Th1 related genes. FIG. 11D provides Violin plot showing the distribution and change of indicated gene expression in FIG. 11B and FIG. 11C. FIG. 11E provides validation of single cell data using flow cytometry to detect CD4⁺ CAR-T cells. FIG. 11F provides analysis of the correlation between tumor growth change and absolute cell number of CAR-expressing CD4 T cells/cm² tumor. *indicates Q<0.05 in the change of percentage expression cells and fold change is greater than 2. # indicates Q<0.05 in the change of cell number. Q value is determined by gene specific analysis (GSA) from Partek flow workstation. * or # indicates change is significant higher for +DMXAA VS −DMXAA. Single cell sequencing data represent 3 mice/treatment group and flow cytometric analysis is pooled from 2 independent experiment for n=8.

FIGS. 12A-12C demonstrate DMXAA treatment enhances anti-tumor function of Th/Tc17 CAR-T cells in an IFN-7-dependent manner. Mice received Th/Tc17 CAR-Ts in the presence or absence of DMXAA therapy. 7 days later, RNA from tumors was isolated and analyzed by microarray for Th17/Th1 response. FIG. 12A provides heat map depicting genes where fold change was significant with a 3-fold increase. Each column represents an individual mouse. FIG. 12B shows quantification of significant changes (P<0.05) in TME for Th17 or Th1 response following DMXAA treatment. FIG. 12C shows mice were treated intraperitoneally with anti-IFN-γ (250 μg/mouse) twice a week after Th/Tc17 CAR-Ts therapy. Tumor growth was measured for 2 weeks after therapy. Significance was determined by Student's t-test. Data from FIG. 12C is from two independent experiments with 4 mice per group.

FIGS. 13A-13D demonstrate T cell exhaustion limits the cumulative beneficial effect of DMXAA on Th/Tc17 CAR-T therapy. T cell cluster selected by Cd3e expression from unsupervised clustering of tumor infiltrating immune cells from 7 days and 10 days after Th/Tc17 CAR+D therapy. FIG. 13A provides t-SNE plot (left) and violin plot of T cells for genes that are associated with T cells exhaustion. FIG. 13B shows validation of PD-1 expression using flow-based method to detect high levels of PD-1 expression on T cells from mice receiving Th/Tc17 CAR-T or mock-T cell therapy. Representative flow cytometry histograms (left), the percentage of CAR-T (right) within the CD4 or CD8 T cells group (n=4/group). FIG. 13C provides t-SNE plot of T cells for genes associated with T cell apoptosis. FIG. 13D provides Violin plot showing the distribution and change of indicated gene expression in FIG. 13C. *indicates Q<0.05 in the change of percentage expression cells and fold change is greater than 2. # indicates Q<0.05 in the change of cell number. Red * or # indicates the change is significant for day 10 vs. day 7. Q value is determined by gene specific analysis (GSA) from Partek flow workstation. The single cell sequencing represents 2-3 mice in each treatment group.

FIGS. 14A-14H demonstrate macrophages and myeloid cells are reduced upon DMXAA treatment, but later replaced by suppressive myeloid cells. Myeloid cells from day 7 are selected as in FIG. 3F. FIG. 14A provides t-SNE plot on M1-like (left) and M2-like (right) related genes. FIG. 14B provides Violin plot showing the distribution and change of indicated gene expression in FIG. 14A. FIG. 14C provides t-SNE and violin plot of cd274 (PD-L1). FIG. 14D provides t-SNE plot on genes expressed in inflammatory-like myeloid cells. FIG. 14E provides Violin plot showing the distribution and change of indicated gene expression in FIG. 14D. FIG. 14F provides t-SNE plot of genes expressed in myeloid-like suppressor cells. FIG. 14G provides Violin plot shows the distribution and change of indicated gene expression in FIG. 14F. Myeloid cells from day 7 and day 10 are selected as in FIG. 3J. FIG. 14H provides t-SNE plot (left) and violin plot (right) of myeloid cells for genes associated with myeloid-like suppressor cells. Single cell sequencing data represent 2-3 mice/treatment group.

FIGS. 15A-15C demonstrate anti-PD-1 and anti-GR-1 mAb does not increase therapeutic benefit of 7/15 CAR-T cells with DMXAA. Mice are treated with anti-GR-1, anti-PD-1, DMXAA and Th/Tc17 CAR-Ts, 7/15 CAR-Ts or mock-Ts as described in FIG. 5A. FIG. 15A shows tumor growth in individual mice receiving therapy (n=5-11 mice per group in FIGS. 15E-15F). FIG. 15B provides Kaplan-Meier survival curve. FIG. 15C provides summary of CAR-T cell accumulation in the spleen. Data shown as mean±s.d. * P<0.05, ** P<0.01, *** P<0.001, **** P<0.0001, significance was determined by Student's t-test or two-way ANOVA when comparing tumor growth change between groups.

FIGS. 16A-16D demonstrate differences in proliferative capacity and generation of central memory in Th17/Tc17 CAR-Ts. Mice received 7/15 CAR+D+aP or Th/Tc17 CAR+D+aP treatment. T cells were isolated from the tumor 7 days after therapy and characterized by flow cytometry. FIG. 16A provides representative plot (left) and summary graph (right) of Ki67 expression from CD4⁺ CAR-T cells with 6-9 mice per group. FIGS. 16B-16C provide assessment of the memory phenotype of CD8⁺ CAR-T cells from the tumor at 7 or 12 days after 7/15 CAR+D+aP or Th/Tc17 CAR+D+aP treatment (n=3-5 mice per group). FIG. 16D provides comparison of memory precursor effector cells of CD8⁺ CAR-T cells within the tumor 7 days after 7/15 CAR+D+aP or Th/Tc17 CAR+D+aP treatment (n=4 mice per group shown). Data shown as mean±s.d. * P<0.05, ** P<0.01, significance was determined by Student's t-test.

FIG. 17 provides a model of the activity of CAR T cells against breast cancer. The use of Th/Tc17 CAR-T cells with enhanced proliferation and memory status (intrinsic modification) along with DMXAA to enhance trafficking and reduce immunosuppression in the TME (extrinsic modification) are critical for the effective anti-tumor response of CAR-T cells against breast cancer (left). CAR-T exhaustion (intrinsic resistance) and reversion back to an immunosuppressive TME state (extrinsic resistance) are associated with loss of CAR T cell function (middle). The addition of anti-PD-1 to overcome exhaustion (intrinsic modification) and the depletion of suppressive myeloid cells with anti-Gr-1 (extrinsic modification) lead to sustained tumor remission (right).

### DETAILED DESCRIPTION OF THE INVENTION

Chimeric antigen receptor T (CAR-T) cells have substantial activity against CD19-expressing B cell malignancies. However, treatment of patients with solid tumors using CAR-T cell therapy has been less than effective for a variety of reasons including suboptimal migration and persistence of CAR-T cells in the tumor microenvironment (TME), impaired function mediated by the immunosuppressive TME, and CAR-T cell exhaustion. Described herein are improved chimeric antigen receptor T (CAR-T) cells and methods of producing the same. Also described herein are methods of treating a disease, e.g., a solid tumor, by administering the improved CAR-T cells.

CAR T cells are produced by obtaining T cells, such as from a subject in need thereof or from a donor subject and manipulating the cells such that they include chimeric antigen receptors (CARs). The CARs provide the ability to target specific proteins on cancer cells and include an antigen recognition domain, an extracellular hinge region, a transmembrane domain, and an intracellular T cell signaling domain. CAR T cells may be classified as first generation, second generation, third generation, or fourth generation. First generation CARs were engineered with only the CD3ζ domain. Second generation CARs were engineered with the CD3ζ domain and a costimulatory signaling domain (e.g., CD28 or 4-1BB). Third generation CARs are engineered to include the CD3ζ (domain in addition to two costimulatory signaling domains (e.g., both CD28 and CD137). Finally, fourth generation CARs, also referred to as T-cells redirected for universal cytokine-mediated killing (TRUCKs) are engineered to include the CD3ζ domain, two costimulatory signaling domains (e.g., both CD28 and CD137), and some additional genetic modification, such as the addition of transgenes for cytokine secretion or additional costimulatory signaling domains.

Described herein are CAR-T cells developed to enhance CAR-T cell function in solid tumors. The CAR-T cells may be second and/or third generation CAR-T cells. In some embodiments, a CAR-T cell includes the costimulatory domain from CD28 or CD137. In one embodiment, a CAR-T cell includes the costimulatory domains from CD28 and CD137. In some embodiments, a CAR-T cell includes the transmembrane domain from CD8. In some embodiments, the CAR-T cells are 7/15 CAR-T cells. In some embodiments, the CAR-T cells are Th/Tc17 CAR-T cells. A population of CAR-T cells may include a roughly equal ratio of CD4⁺ to CD8⁺ cells. In some aspects, the CD4⁺ and CD8⁺ T cells are present in a ratio of about 2:1, 1.75:1, 1.5:1, 1.25:1, 1:1, 1:1.25, 1:1.5, 1:1.75, or 1:2.

Disclosed herein are methods of treating a disease comprising administering to a subject a chimeric antigen receptor modified T cell (CAR T cell). The CAR T cells are administered to the subject to target and destroy cancer cells. In some embodiments, the CAR T cell is a second or third generation CAR T cell. In some embodiments, a CAR T cell is a Th/Tc17 CAR T cell or a 7/15 CAR T cell. In certain embodiments, the CAR T cells are Th/Tc17 CAR T cells. Th/Tc17 CAR T cells may exhibit superior persistence in a tumor as compared to other CAR T cells, such as IL7/15 CAR T cells. In some embodiments, the Th/Tc17 CAR T cells are engineered to include the costimulatory domain from CD28. In some embodiments, the CAR construct for Th/Tc17 CAR T cells includes a scFv (7.16.4), hinge and transmembrane domain from CD8, and intracellular domains from CD28 and CD3ζ. In some embodiments, the CAR T cells are either derived from T cells removed from a subject in need thereof, e.g., are autologous, or are derived from T cells removed from a healthy donor, e.g., are allogenic. In some aspects, T cells are engineered from stem cells.

In some embodiments, a CAR T cell is administered to a subject, e.g. in a subject in need thereof, such as a subject diagnosed with cancer, in combination with a STING agonist. A STING agonist activates the STING pathway, which, in some aspects, can cause a tumor to go from “cold” to “hot”. A “cold tumor” is used herein to refer to a tumor that does not provoke an immune response, while a “hot tumor” is used herein to refer to a tumor that is more prone to recognition by the immune system. In some embodiments, a STING agonist increases expression of CXCL9 and CXCL10 by myeloid cells located within the tumor microenvironment.

In some embodiments, a STING agonist is any viral or bacterial product that activates the STING pathway. In certain embodiments, a STING agonist is selected from the group consisting of DMXAA, cGAMP, CMA, c-Di-AMP, and c-Di-GMP. In certain embodiments, a STING agonist is DMXAA. A STING agonist may be administered prior to administration of a CAR T cell, concurrently with a CAR T cell, or after administration of a CAR T cell. In certain embodiments, a STING agonist is administered prior to administration of the CAR T cells. In some aspects, administration of a STING agonist in combination with a CAR T cell enhanced the persistence of the CAR T cells in the tumor microenvironment (TME). In certain aspects, administration of a STING agonist increased antitumor efficacy and/or overall survival of the CAR T cells.

In some embodiments, a myeloid-derived suppressor cell (MDSC) depleting agent is administered to the subject in combination with the CAR T cells, e.g., Th/Tc17 CAR T cells engineered to include a CD28 costimulatory domain, and the STING agonist, e.g., DMXAA. An MDSC depleting agent may be a MDSC targeting agent. In some aspects, an MDSC depleting agent is an antibody, such as an anti-Gr-1 antibody. In some aspects, MDSC is modified, e.g., depleted, by an agent that leads to differentiation or maturation of the MDSCs. Non-limiting examples of agents that lead to differentiation or maturation of the MDSCs include retinoids, chemotherapy drugs (e.g., gemcitabine), and antibodies that inhibit the M-CSF, g-CSF, or GM-CSF receptors. In other aspects, the migration of MDSCs to the tumor environment is diminished by inhibiting the function of CXCL2, CXCL1, or CXCL5. In still other aspects, the function of MDSCs is targeted by inhibiting the activity of proteins important for the function of these cells, such as Cox-2, PGE2, arginase I/II, or iNOS. In some embodiments, the MDSC depleting or targeting agent is administered concurrently with CAR T cells and/or STING agonist. In other embodiments, the MDSC depleting agent is administered sequentially with the CAR T cells and/or STING agonist. In some aspects, an MDSC depleting agent is administered concurrently with CAR T cells. In some aspects, an MDSC depleting agent is administered within the first two weeks following CAR T cell therapy. In some aspects, an MDSC depleting agent is administered concurrently with a STING agonist. In some aspects, a STING agonist, e.g., DMXAA, is administered to a subject, followed by administration of a population of CAR T cells, e.g., Th/Tc17 CAR T cells engineered to include a CD28 costimulatory domain, and further followed by administration of an MDSC depleting agent, e.g., anti-Gr-1 antibody. In certain aspects, administration of an MDSC depleting agent to a subject in combination with CAR T cells and a STING agonist improves overall survival of the subject and improves control of tumor growth.

In some embodiments, a PD-1 inhibitor is administered to the subject in combination with the CAR T cells and the STING agonist. In certain embodiments, the PD-1 inhibitor is administered to the subject in combination with the MDSC depleting agent, e.g., anti-Gr-1 antibody, a population of CAR T cells, e.g., Th/Tc17 CAR T cells engineered to include a CD28 costimulatory domain, and the STING agonist, e.g., DMXAA. In some aspects, a PD-1 inhibitor is an anti-PD-1 antibody or a fusion protein targeting PD-1. In some embodiments, PD-1 is inactivated using gene editing in the CAR T cell. In certain embodiments, PD-1 is inactivated via CRISPR/Cas9, TALENs, or ZFN in the CAR T cell. In some embodiments, a PD-1 inhibitor, is administered concurrently with the MDSC depleting agent, CAR T cells and/or the STING agonist. In other embodiments, the PD-1 inhibitor is administered prior to the MDSC targeting agent, and in some aspects is administered concurrently with or, alternatively after, CAR T cell therapy. In other embodiments, the PD-1 inhibitor is administered sequentially with the MDSC depleting agent, the CAR T cells, and the STING agonist. In some aspects, the PD-1 inhibitor, e.g., anti-PD-1 antibody, is administered to a subject concurrently with an MDSC depleting agent, e.g., anti-Gri-1 antibody. In some aspects, a STING agonist, e.g., DMXAA, is administered to a subject, followed by administration of a population of CAR T cells, e.g., Th/Tc17 CAR T cells engineered to include a CD28 costimulatory domain, and further followed by administration of an MDSC depleting agent, e.g., anti-Gr-1 antibody, and a PD-1 inhibitor, e.g., anti-PD-1 antibody. In certain aspects, administration of a PD-1 inhibitor to a subject in combination with CAR T cells and a STING agonist improves overall survival of the subject and improves control of tumor growth.

In some embodiments, a subject is further administered an anti-IL-6 antibody, an anti-IL-6R antibody, or an antibody or protein targeting IL-1. In certain embodiments, the anti-IL-6 antibody is administered to the subject after administration of the STING agonist, the population of CAR T cells, the MDSC depleting agent, and the PD-1 inhibitor. In some aspects, the anti-IL-6 antibody is administered at the peak time of symptoms from CRS, or about 3 to 7 days, or about 5 days after administration of the STING agonist, the population of CAR T cells, the MDSC depleting agent, and the PD-1 inhibitor.

In some embodiments, a subject has a disease or disorder. In certain embodiments, the disease is cancer, which term is generally used interchangeably to refer to a disease characterized by one or more tumors, e.g., one or more malignant or potentially malignant tumors. The term “tumor” as used herein encompasses abnormal growths comprising aberrantly proliferating cells. As known in the art, tumors are typically characterized by excessive cell proliferation that is not appropriately regulated (e.g., that does not respond normally to physiological influences and signals that would ordinarily constrain proliferation) and may exhibit one or more of the following properties: dysplasia (e.g., lack of normal cell differentiation, resulting in an increased number or proportion of immature cells); anaplasia (e.g., greater loss of differentiation, more loss of structural organization, cellular pleomorphism, abnormalities such as large, hyperchromatic nuclei, high nuclearxytoplasmic ratio, atypical mitoses, etc.); invasion of adjacent tissues (e.g., breaching a basement membrane); and/or metastasis. The term “tumor” includes malignant solid tumors, e.g., carcinomas (cancers arising from epithelial cells), sarcomas (cancers arising from cells of mesenchymal origin), and malignant growths in which there may be no detectable solid tumor mass (e.g., certain hematologic malignancies). Cancer includes, but is not limited to: breast cancer; biliary tract cancer; bladder cancer; brain cancer (e.g., glioblastomas, medulloblastomas); cervical cancer; choriocarcinoma; colon cancer; endometrial cancer; esophageal cancer; gastric cancer; hematological neoplasms including acute lymphocytic leukemia and acute myelogenous leukemia; T-cell acute lymphoblastic leukemia/lymphoma; hairy cell leukemia; chronic lymphocytic leukemia, chronic myelogenous leukemia, multiple myeloma; adult T-cell leukemia/lymphoma; intraepithelial neoplasms including Bowen's disease and Paget's disease; liver cancer; lung cancer; lymphomas including Hodgkin's disease and lymphocytic lymphomas; neuroblastoma; melanoma, oral cancer including squamous cell carcinoma; ovarian cancer including ovarian cancer arising from epithelial cells, stromal cells, germ cells and mesenchymal cells; neuroblastoma, pancreatic cancer; prostate cancer; rectal cancer; sarcomas including angiosarcoma, gastrointestinal stromal tumors, leiomyosarcoma, rhabdomyosarcoma, liposarcoma, fibrosarcoma, and osteosarcoma; renal cancer including renal cell carcinoma and Wilms tumor; skin cancer including basal cell carcinoma and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullary carcinoma. It will be appreciated that a variety of different tumor types can arise in certain organs, which may differ with regard to, e.g., clinical and/or pathological features and/or molecular markers. Tumors arising in a variety of different organs are discussed, e.g., the WHO Classification of Tumours series, 4^{th} ed, or 3^{rd} ed (Pathology and Genetics of Tumours series), by the International Agency for Research on Cancer (IARC), WHO Press, Geneva, Switzerland, all volumes of which are incorporated herein by reference.

In certain embodiments, a subject is administered a therapeutic regimen comprising a population of CAR T cells, e.g., Th/Tc17 CAR T cells engineered to include a CD28 costimulatory domain, a STING agonist, e.g., DMXAA, an MDSC depleting agent, e.g., anti-Gr-1 antibody, and a PD-1 inhibitor, e.g., anti-PD-1 antibody, to treat a cancer, e.g., a cancer characterized by one or more tumors. In some aspects, the administration of the therapeutic regimen to the subject results in the subject suffering from cytokine-release like syndrome (CRS). A subject suffering from CRS may further be administered an anti-IL-6 antibody.

The agents, e.g., the population of CAR T cells, STING agonist, MDSC depleting agent, PD-1 inhibitor, and/or anti-IL-6 antibody, may be administered in pharmaceutically acceptable solutions, which may routinely contain pharmaceutically acceptable concentrations of salt, buffering agents, preservatives, compatible carriers, adjuvants, and optionally other therapeutic ingredients.

The agents may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

In some embodiments, agents may be administered directly to a tissue. Direct tissue administration may be achieved by direct injection. The agents may be administered once, or alternatively they may be administered in a plurality of administrations. If administered multiple times, the peptides may be administered via different routes. For example, the first (or the first few) administrations may be made directly into the affected tissue while later administrations may be systemic.

For oral administration, compositions can be formulated readily by combining the agent with pharmaceutically acceptable carriers well known in the art. Such carriers enable the agents to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration. For buccal administration, the compositions may take the form of tablets or lozenges formulated in conventional manner.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated in some embodiments to achieve appropriate systemic levels of compounds.

Specific examples of certain aspects of the inventions disclosed herein are set forth below in the Examples.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, cell, or non-human transgenic animal, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### Example 1

### Results

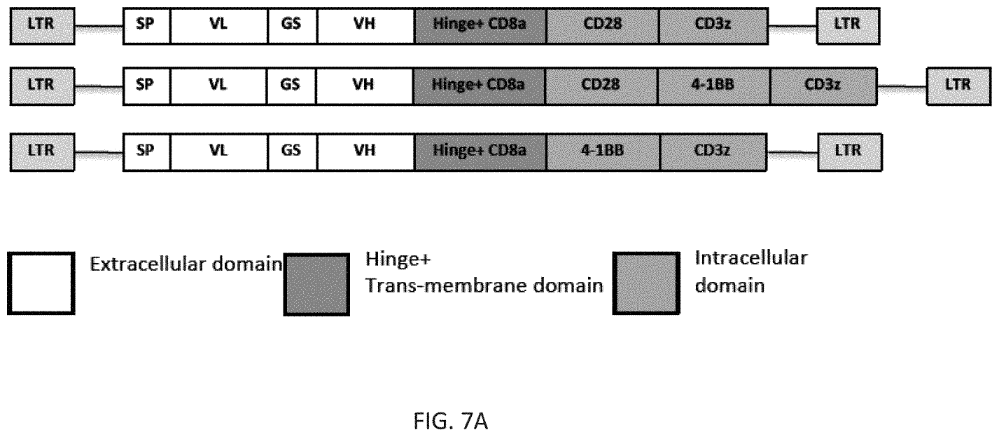

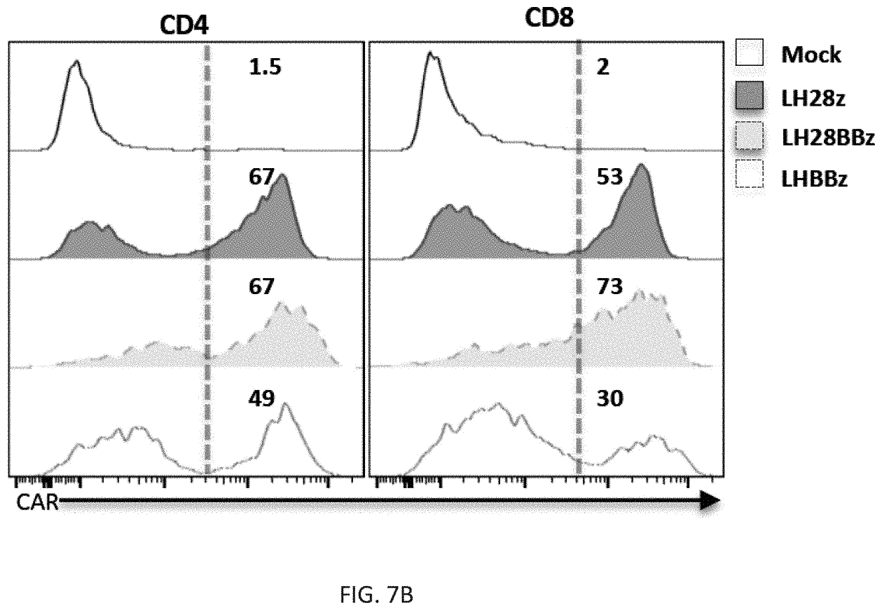

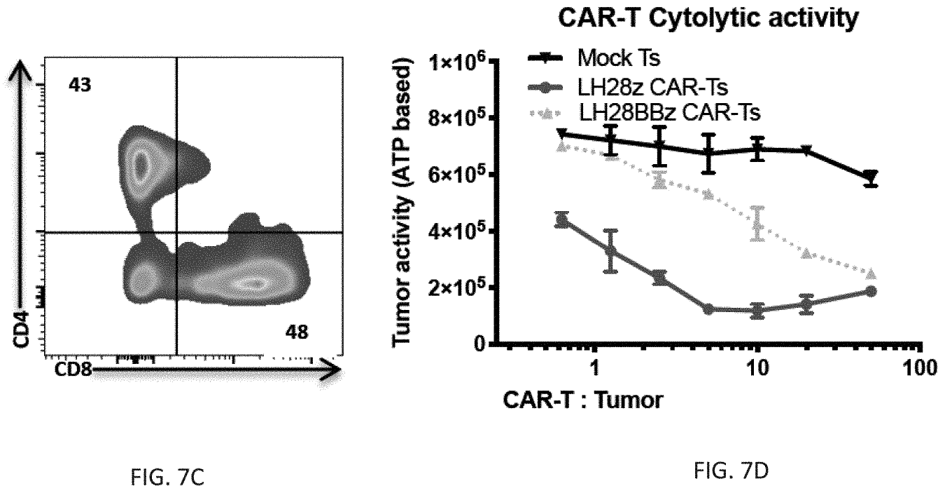

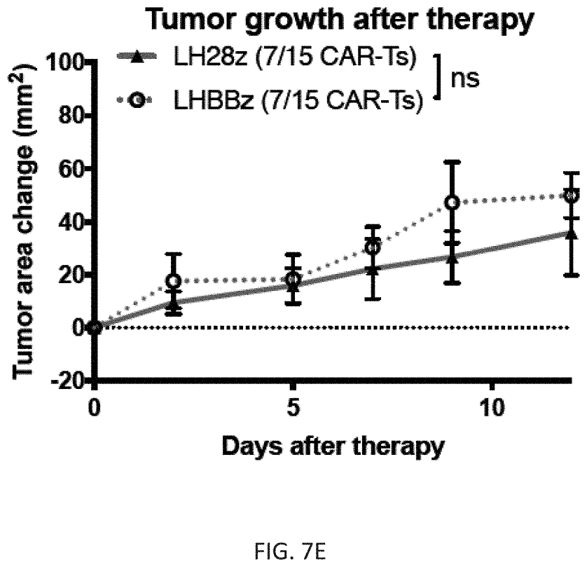

Development of an Orthotopic Immune Competent Model to Evaluate CAR T Cell Therapy Given the need to understand mechanistically how to enhance CAR T cell function in solid tumors, second and third generation murine CAR constructs were developed targeting the proto-oncogene Neu (FIG. 1A, FIG. 7A). Second generation CAR T cells were generated using the co-stimulatory domains from either CD28 (termed LH28z) or CD137 (4-1BB termed LHBBZ). Third generation CAR T cells were generated using both CD28 and CD137 signaling endodomains (LH28BBZ). Neu-specific CAR T cells were transduced and expanded with IL-7 and IL-15 (7/15 CAR T cells) (FIG. 1B, FIG. 7B) at an equal ratio of CD4⁺ to CD8⁺ T cells (FIG. 7C). These cells were functional in vitro as they killed Neu-expressing target cells (FIG. 1C and FIG. 7D) and released pro-inflammatory cytokines only in response to Neu⁺ tumor cells (FIG. 1D). However, it was consistently found that the transduction efficiency of the LHBBz construct was inferior to LH28z or LH28BBBz constructs (FIG. 7B). Furthermore, both in vitro (FIG. 7C) and in vivo experiments (FIG. 7D) showed inferior antitumor activity of LH28BBz and LHBBz CAR T cells as compared to LH28z CAR T cells. Given these findings, the focus for the remainder of this work was on enhancing the function of LH28z CAR T cells.

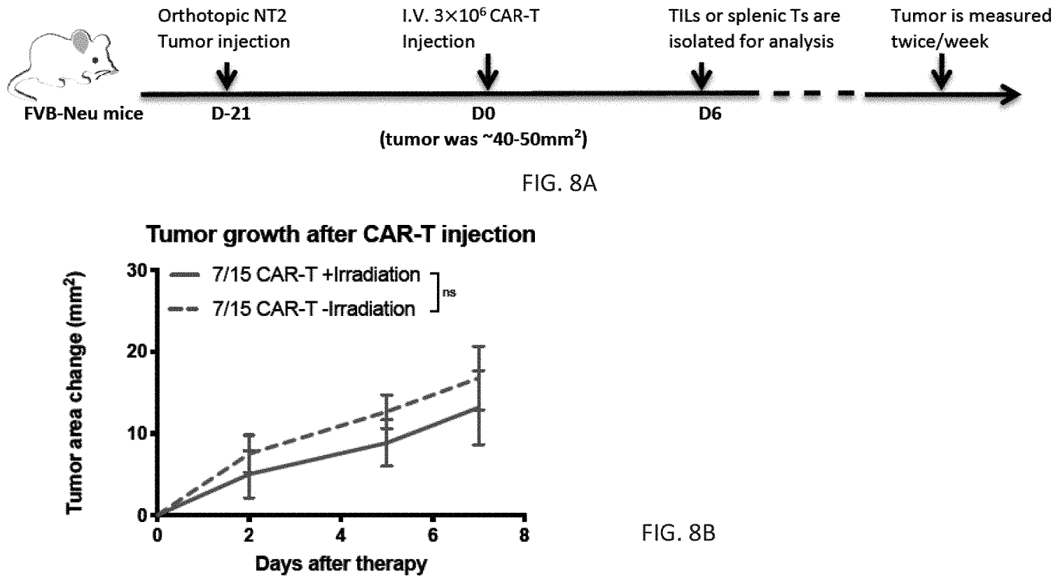

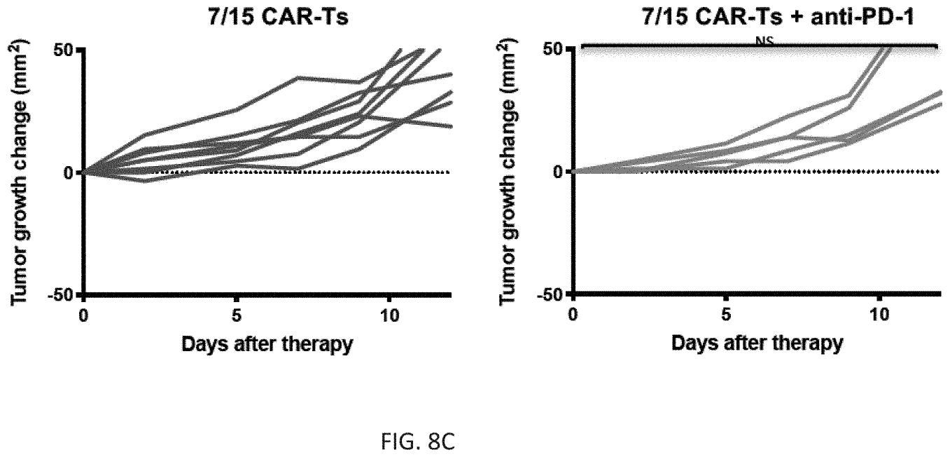

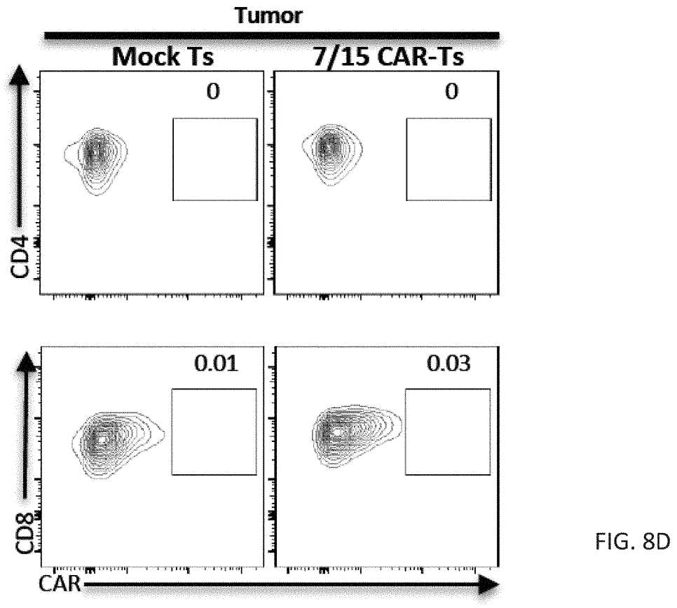

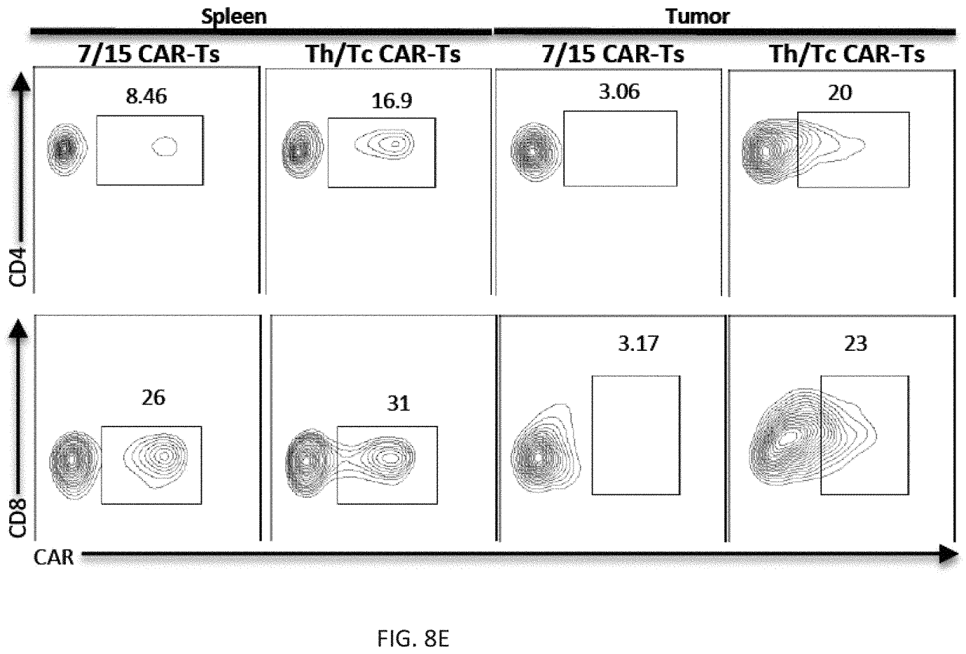

The antitumor effects of CAR T cells were evaluated in an orthotopic murine breast tumor model of locally advanced disease in immunocompetent mice (FIG. 8A). Despite their in vitro cytolytic function, LH28z 7/15 CAR T cells were not able to control tumor growth in vivo (FIG. 1E), even if hosts were lymphodepleted using irradiation prior to CAR T cell infusion (FIG. 8B). Combination of 7/15 LH28z CAR T cells with an anti-PD-1 mAb did not improve the outcome (FIG. 8C) indicating that blocking the PD-1/PDL-1 pathway is insufficient for tumor control by 7/15 LH28z CAR T cells. Furthermore, very few CAR T cells were detected in the TME 5 days post treatment (FIG. 8D), suggesting that the limited efficacy of 7/15 LH28z CAR T therapy was at least partly due to poor trafficking and persistence of CAR T cells in the TME.

Th/Tc 17 CAR T Cells have Enhanced In Vivo Persistence

Th17 cells and their counterpart Tc17 cells (CD8⁺ T cells that express IL-17A) have longer persistence in the TME, in part due to their better survival in vivo^{5,6}. To evaluate if Th/Tc17 CAR T cells had better migration and/or persistence, LH28z Th/Tc17 CAR T cells were generated (termed Th/Tc17 CAR T cells). These cells were compared with 7/15 CAR T cells. Transduction efficacy (FIG. 1F) and killing activity in vitro (FIG. 1G) were comparable. Th/Tc17 CAR T cells released IL-17A and TNF, while 7/15 CAR T cells expressed IFN-7 and TNF when co-cultured with Neu⁺ tumor cells in vitro (FIG. 1H).

Next, the in vivo antitumor effects of Th/Tc17 CAR T cells were compared to those of 7/15 CAR T cells. Th/Tc17 CAR T cells were superior to 7/15 CAR T cells in controlling tumor growth up to day 5 post-infusion (FIG. 1I). To identify mechanisms associated with the improved early efficacy of Th/Tc17 CAR T cells, tumor-infiltrating lymphocytes (TILs) were isolated from the TME and their phenotype was assessed 5 days after therapy. There was a significant increase in both CD4⁺ and CD8⁺ CAR T cells in the tumor and CD4⁺ CAR-T cells in the spleen of mice receiving Th/Tc17 CAR T cells compared to the recipients of 7/15 CAR T cells (FIG. 1J and FIG. 8E). However, Th/Tc17 CAR T cells were only modestly increased in the TME, which may in part explain the lack of long-term tumor control. Thus, Th/Tc17 CAR T cells exhibit modestly improved migration/persistence to the TME and improved early tumor control, but this did not lead to a long-term anti-tumor response.

### The STING Agonist DMXAA Greatly Enhances CAR T Efficacy

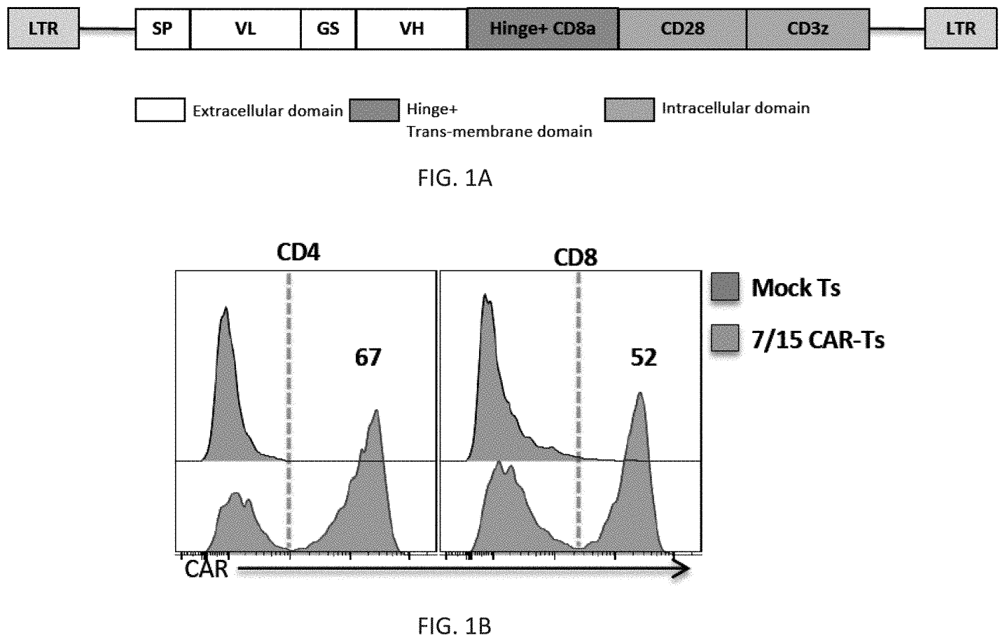

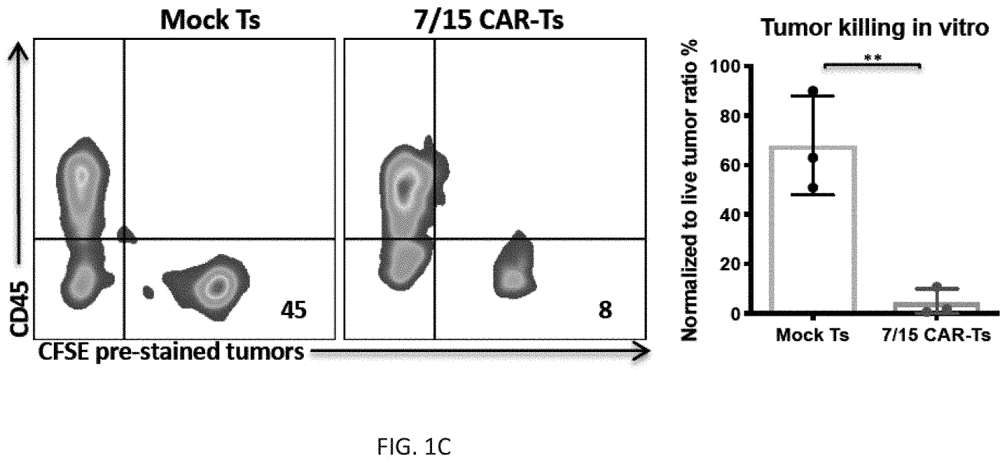

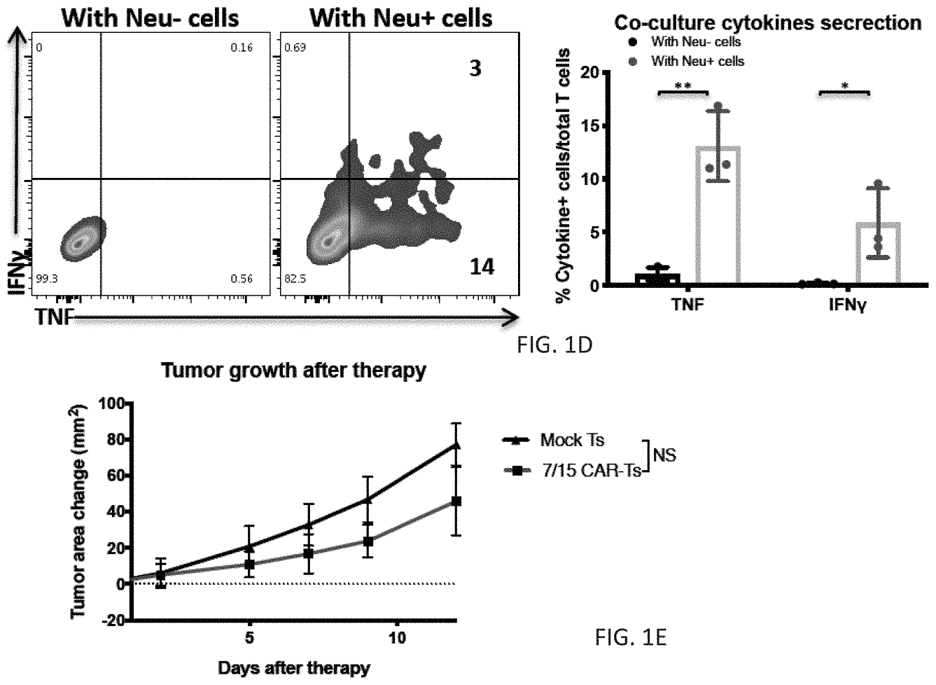

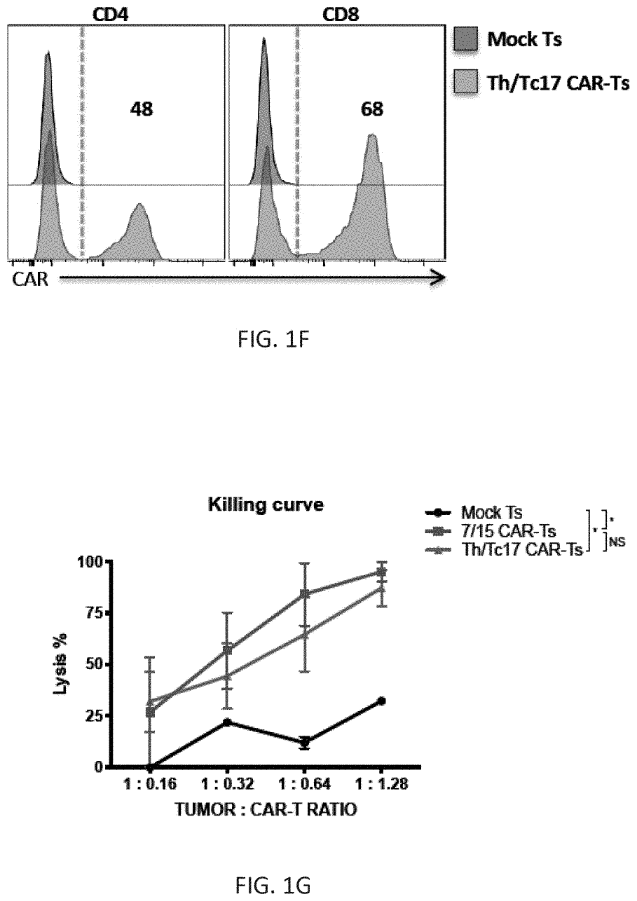

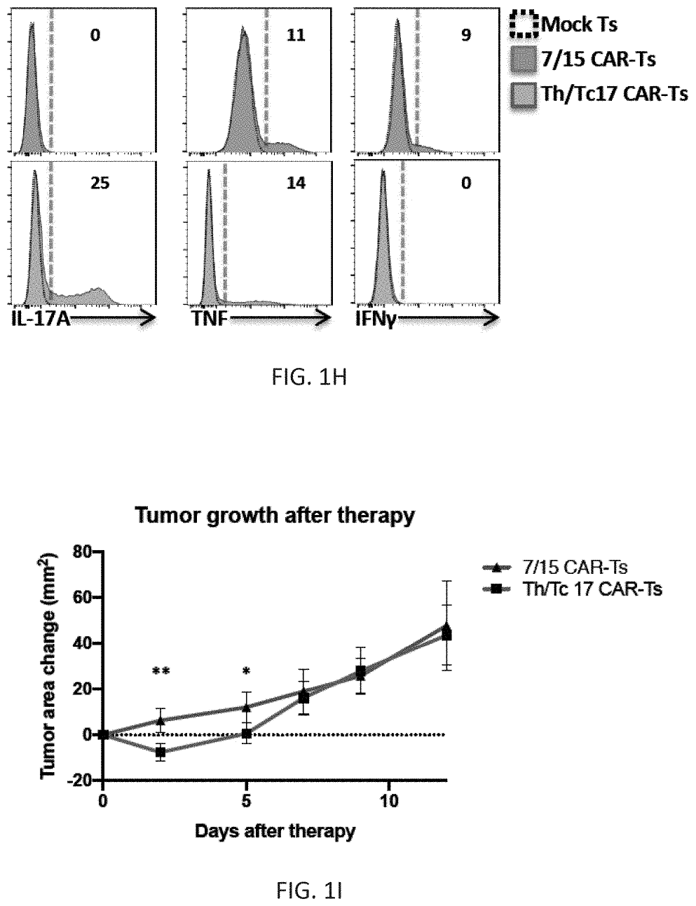

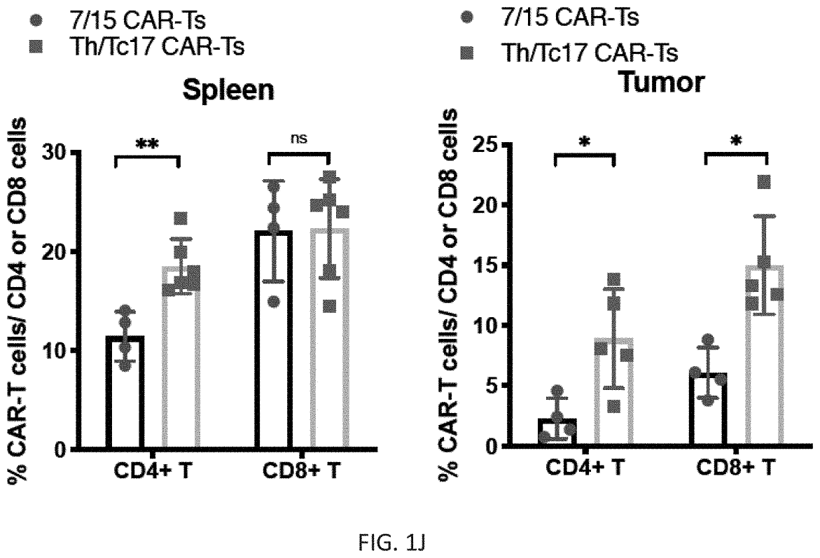

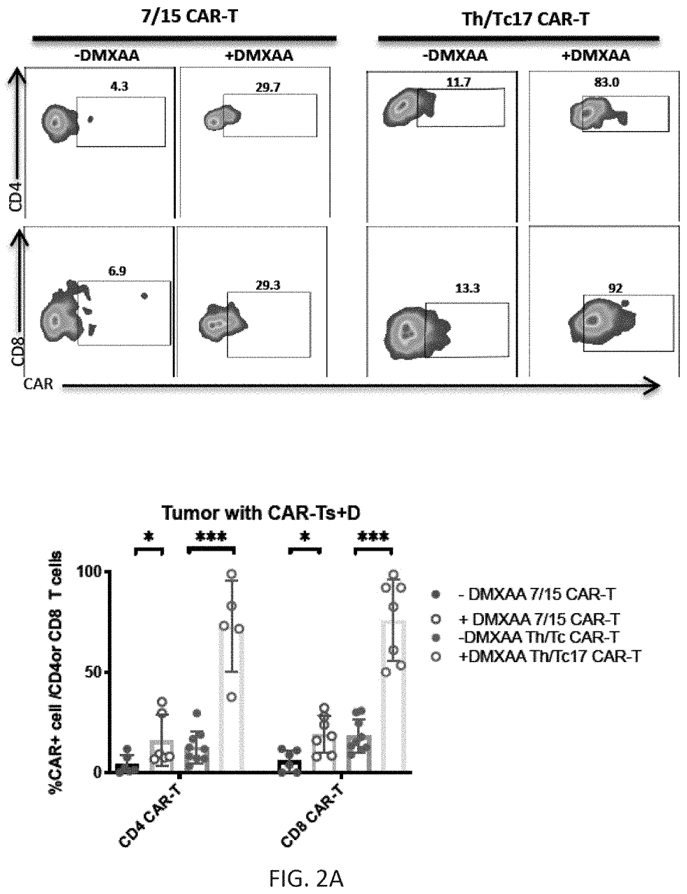

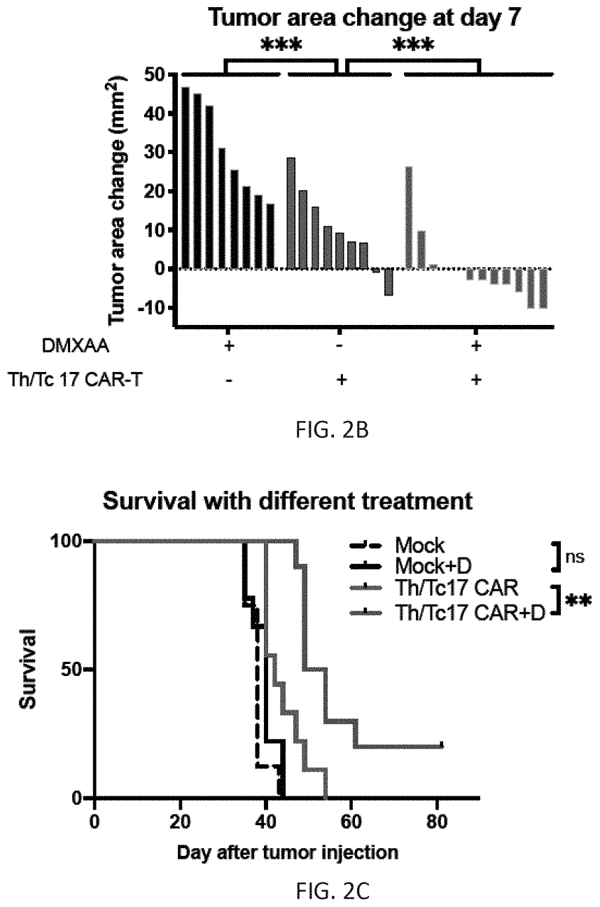

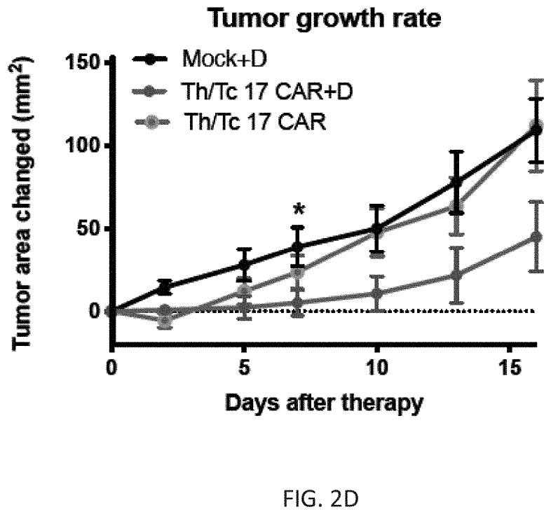

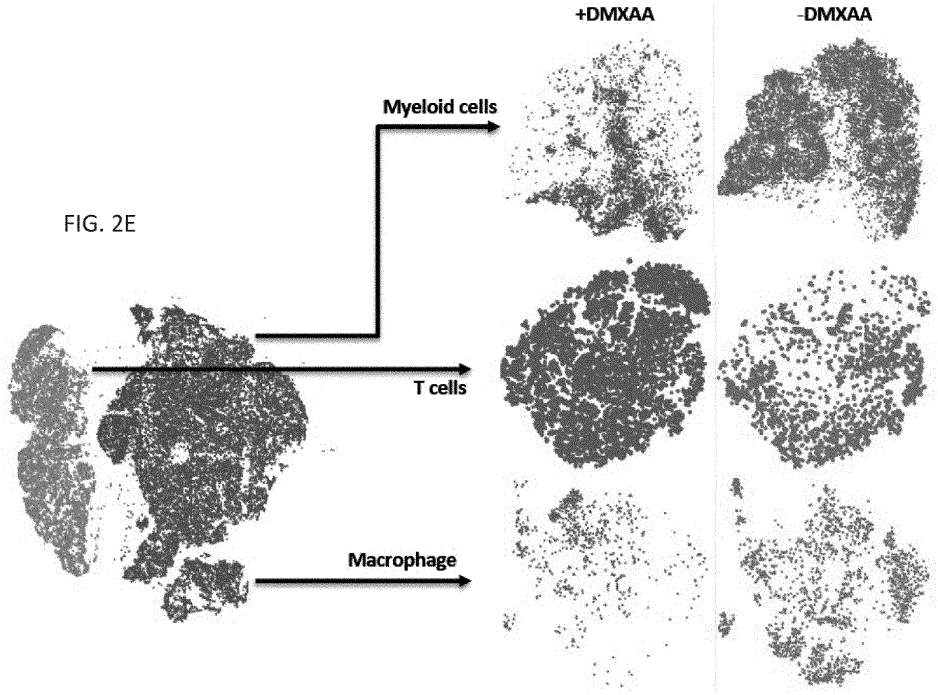

Given the short-term efficacy of Th/Tc17 CAR T cells in vivo, an approach that would further enhance the migration and persistence of CAR T cells in the TME was sought. Recent studies have shown a critical role for the STING pathway in generating “warm” tumors in the setting of checkpoint inhibitor therapy⁹. Thus, the activity of the STING agonist, DMXAA, was evaluated in combination with CAR-T cells. Mice were injected with NT2 tumors, and when tumors reached approximately 50 mm², mice were given 7/15 or Th/Tc17 CAR T cells with DMXAA, which was given at a site remote from the tumor. Interestingly, DMXAA enhanced the persistence of 7/15 CAR T cells in the TME (FIG. 2A). However, this was a modest increase in comparison to the increase when combined with Th/Tc17 CAR T cells. Thus, Th/Tc17 CAR T cells were focused on. The administration of DMXAA prior to Th/Tc17 CAR-T cell infusion, significantly improved anti-tumor efficacy (FIG. 2B) and overall survival (FIG. 2C) compared to mock transduced T cells or Th/Tc17 CAR-T cells alone.

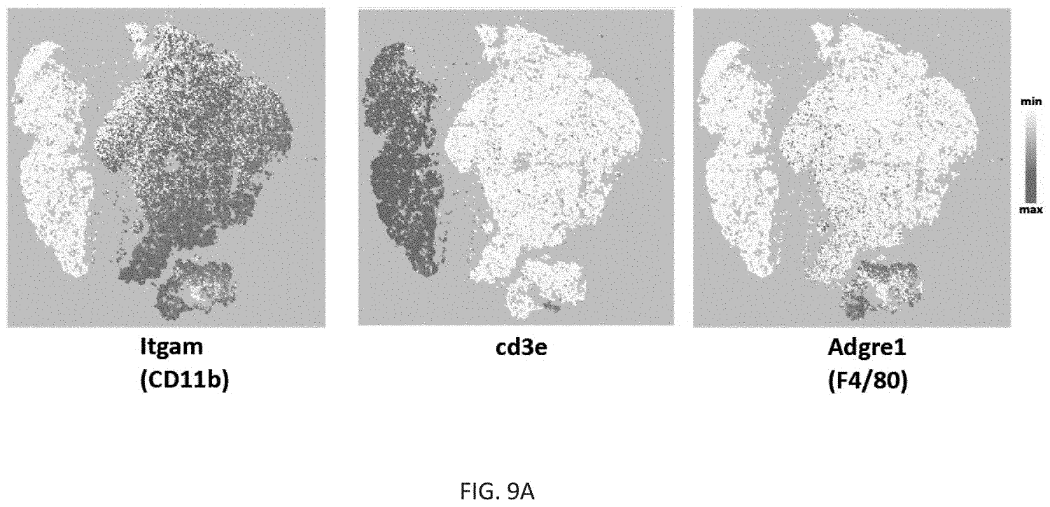

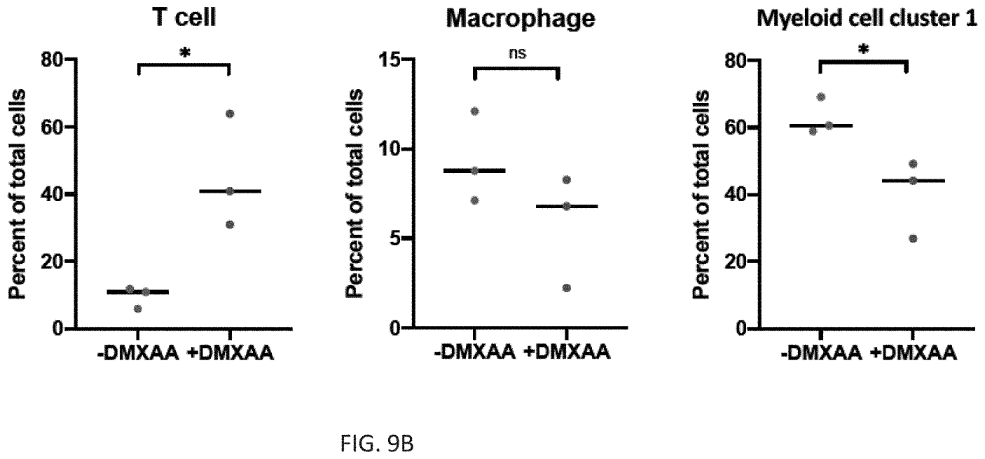

Single cell transcriptome sequencing (scRNA-seq) was performed on CD45⁺ cells isolated from the TME of mice that received Th/Tc17 CAR-T with or without DMXAA. These evaluations were performed on day 7 post CAR T cell therapy, a time point where the greatest tumor control was observed, and at day 10 when tumors began to regrow (FIG. 2D). Based on gene expression profiles, three immune cell populations were identified in the TME in mice receiving Th/Tc17 CAR T cells with or without DMXAA (FIG. 9A). Specifically, at day 7 post-therapy, clusters of T cells, macrophages and a second myeloid cell cluster were identified that each showed differential gene expression profiles in the presence or absence of DMXAA treatment (FIG. 2E). In addition, an increase in T cells and a decrease in a sub-cluster of myeloid cells was noted in the TME on day 7 post CAR T cell therapy (FIG. 9B).

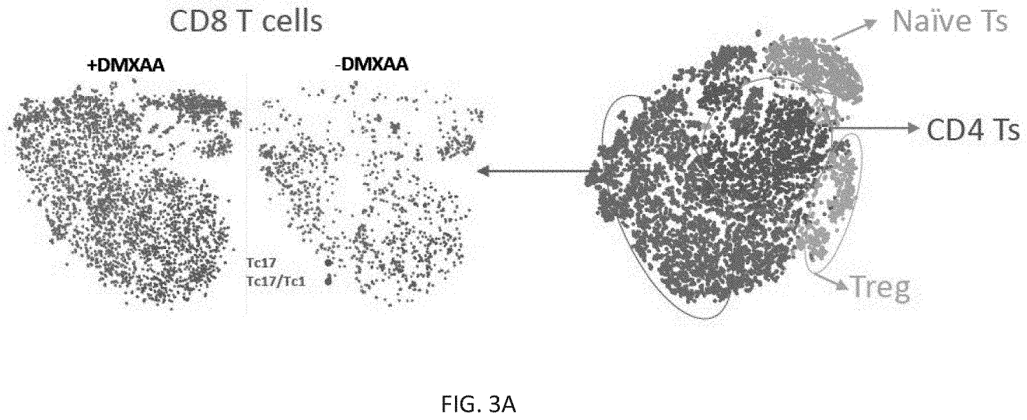

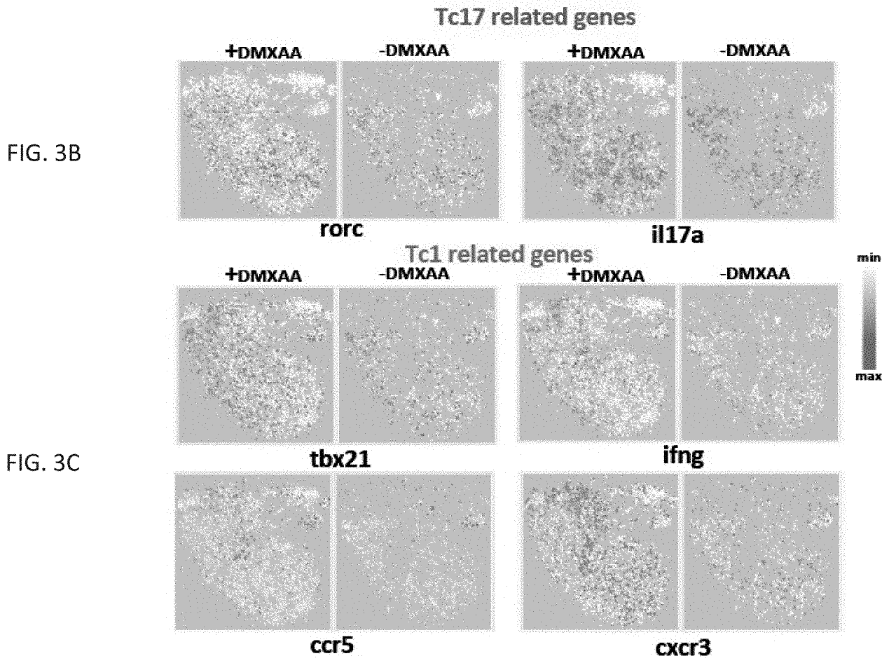

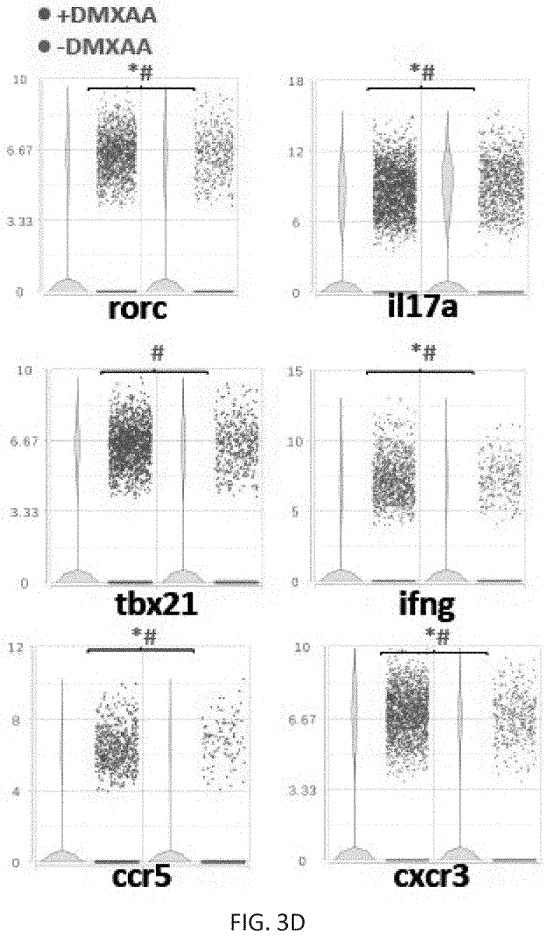

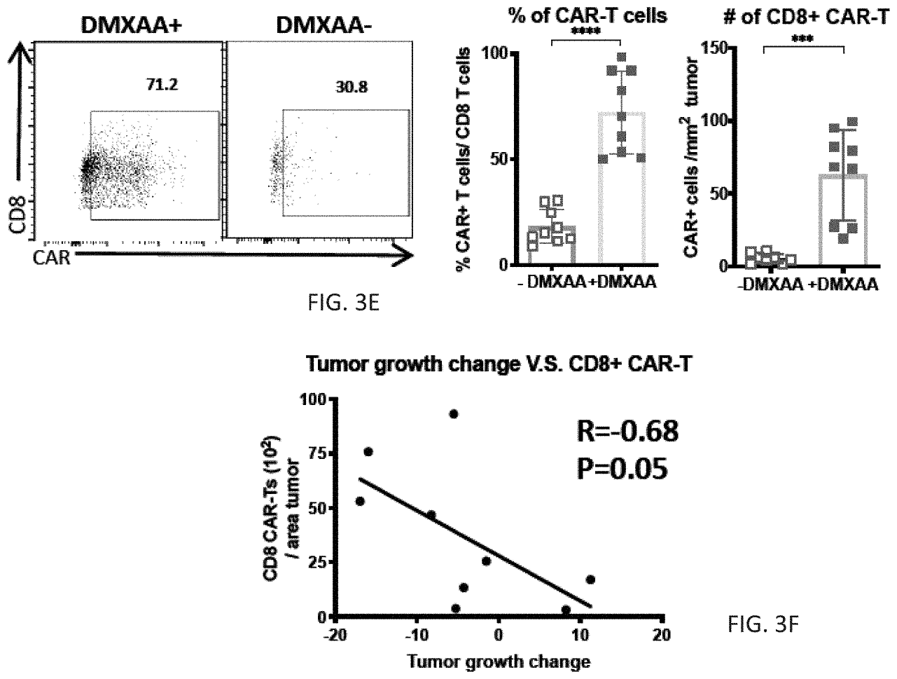

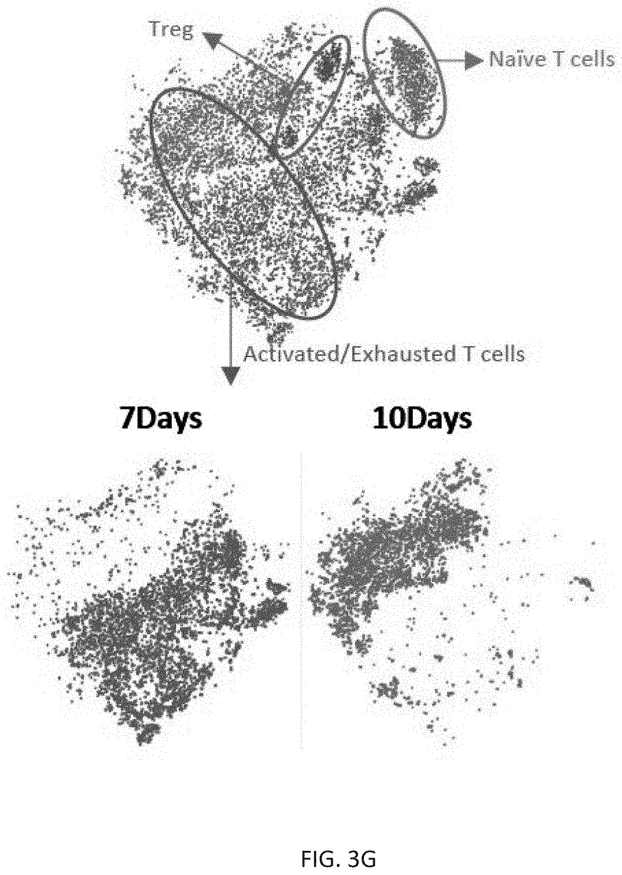

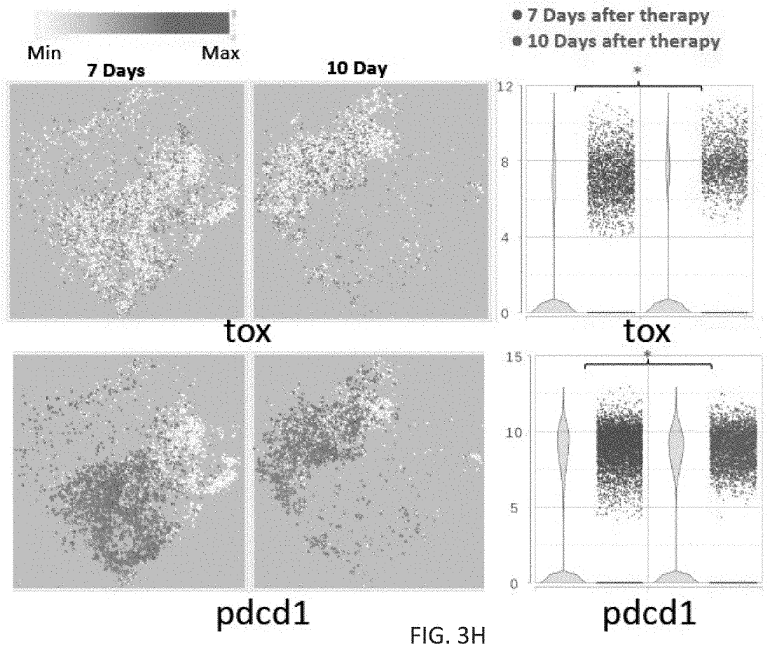

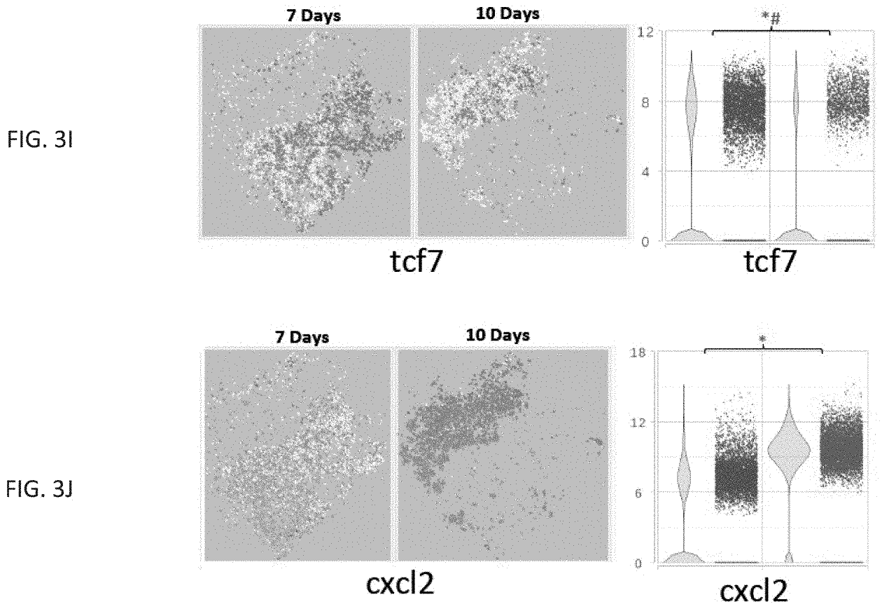

The most noticeable difference in the TME in mice receiving DMXAA was a substantial increase in CAR T cells compared to animals given Th/Tc17 CAR T cells without DMXAA (FIG. 3A). CD8⁺ CAR T cells infiltrating the TME exhibited two different phenotypes. A subset expressed the transcription factor RORC and the cytokine IL17A (FIGS. 3B, 3D) consistent with Tc17 cells. Interestingly, DMXAA treatment enhanced the accumulation of T cells that co-expressed the transcription factor TBX21, the pro-inflammatory cytokine IFNG, the interferon-inducible chemokine receptor CXCR3, and CCR5 a trafficking receptor found on Th1/Tc1 cells (FIGS. 3C-3D). This expression pattern is consistent with the presence of pro-inflammatory Tc1 cells converted from Tc17 cells in the presence of DMXAA. To confirm the increase in CAR T cells in mice receiving DMXAA, flow cytometry was performed to detect CAR T cells in the TME. DMXAA treatment greatly increased the percentage and number of CAR T cells in the TME (FIG. 3E). In addition, a strong association was found between the number of CD8⁺ CAR T cells in the TME and tumor size in mice receiving DMXAA (FIG. 3F).

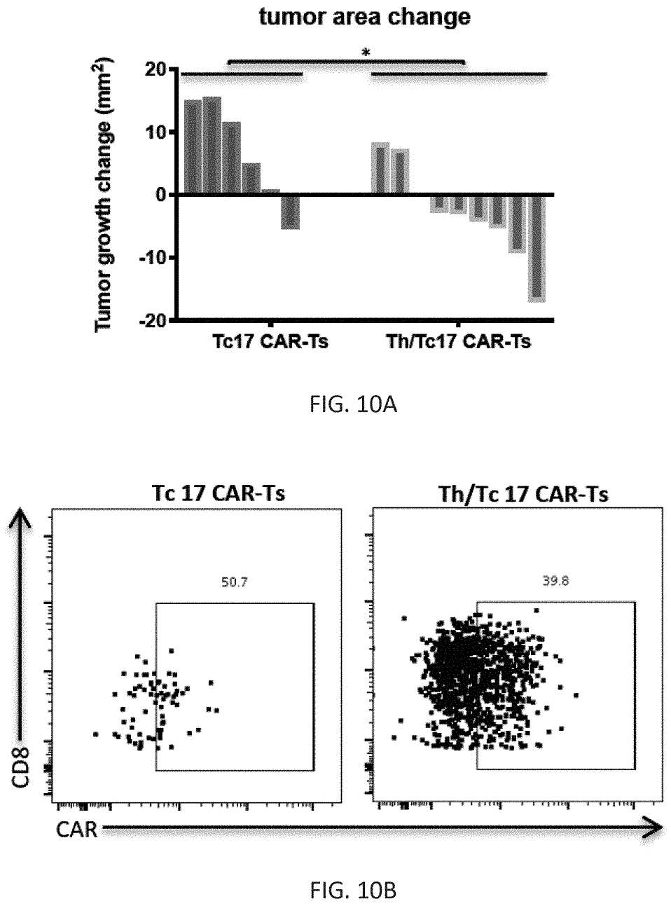

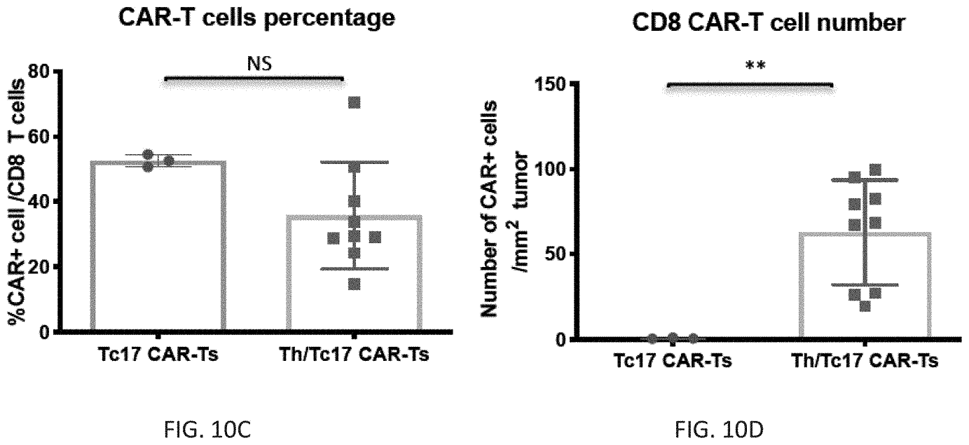

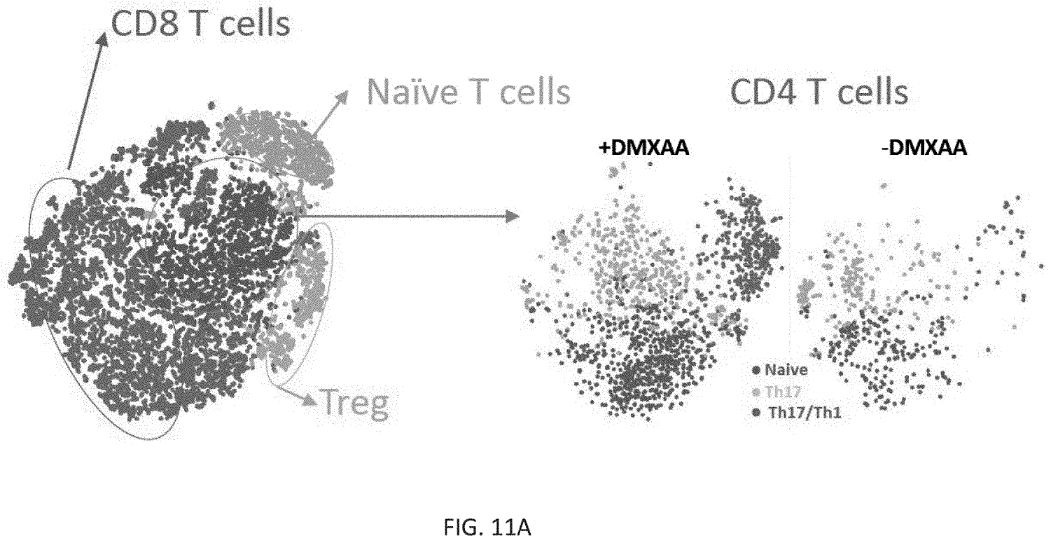

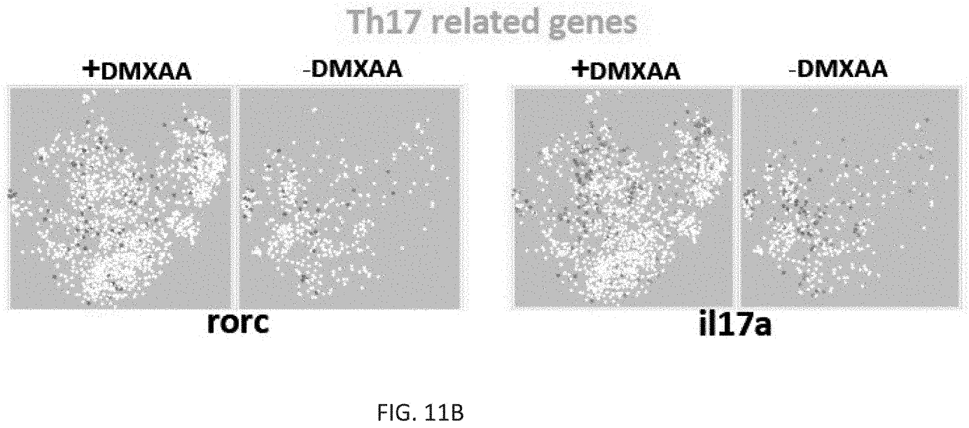

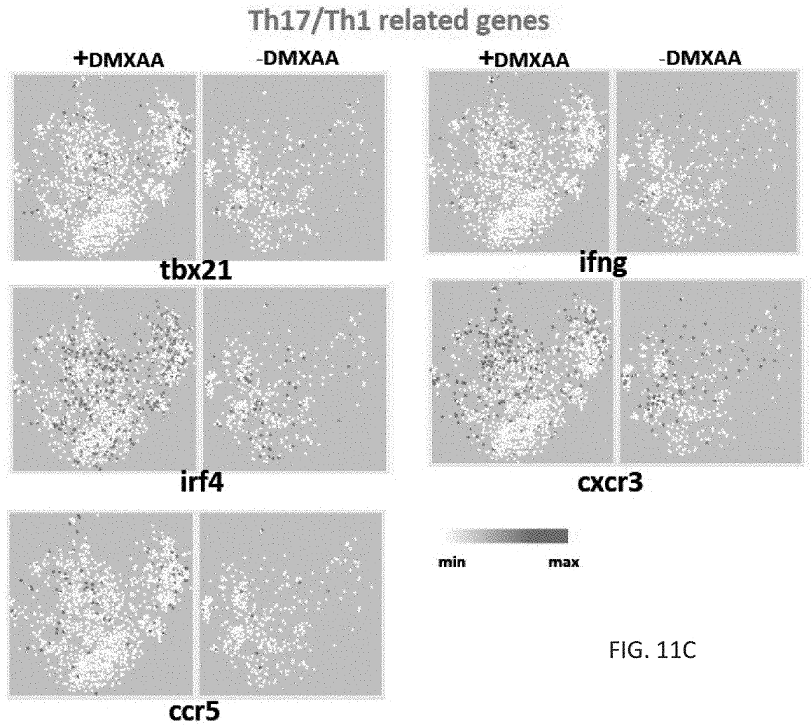

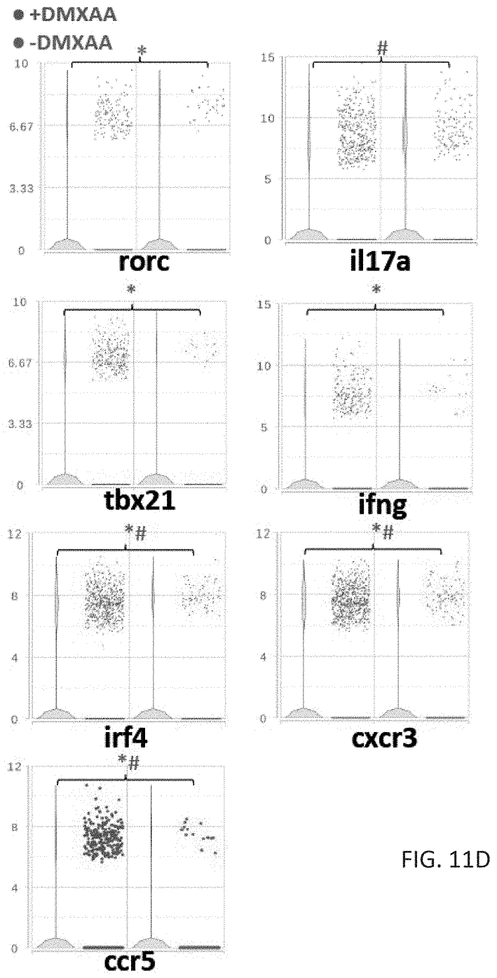

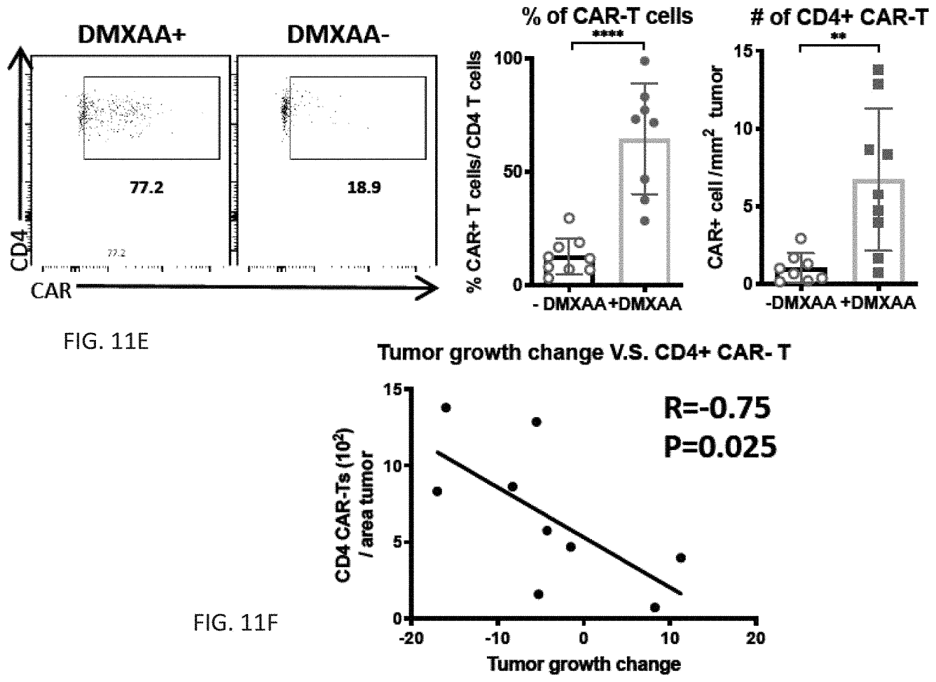

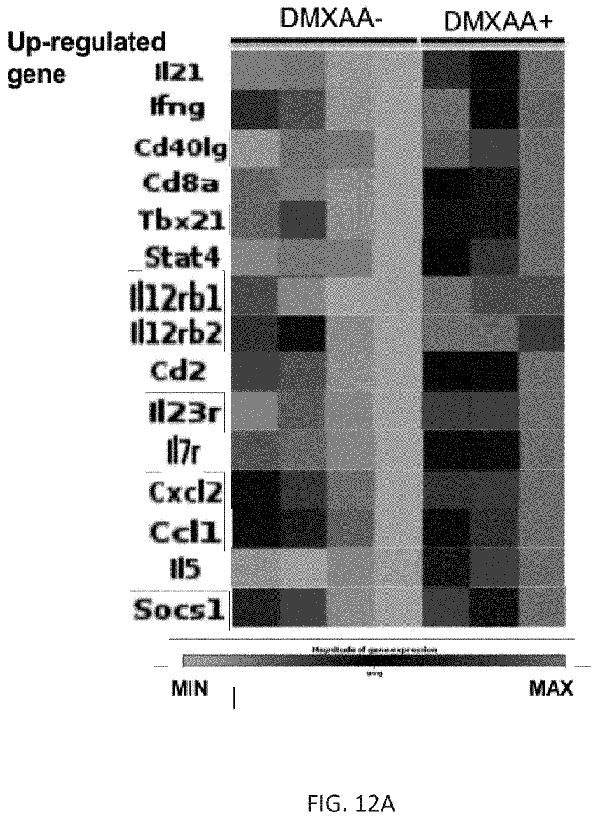

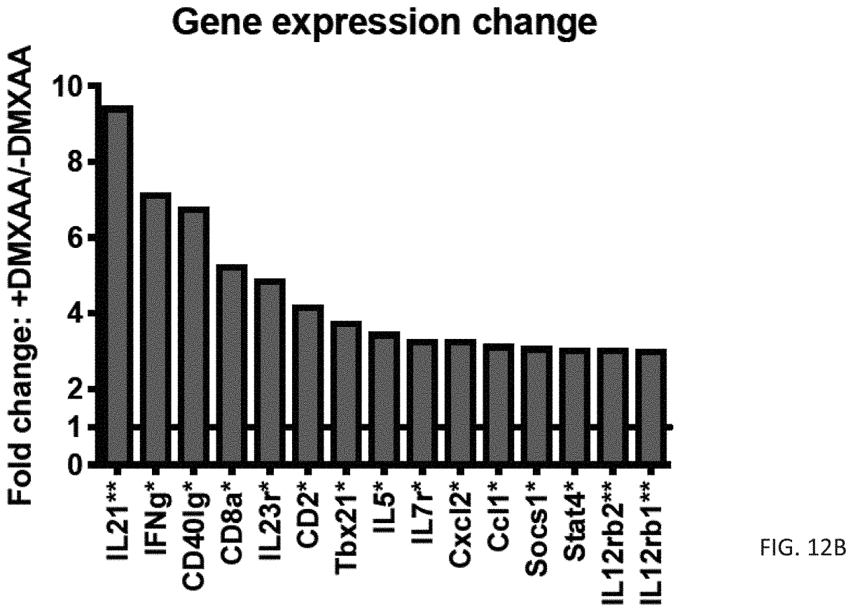

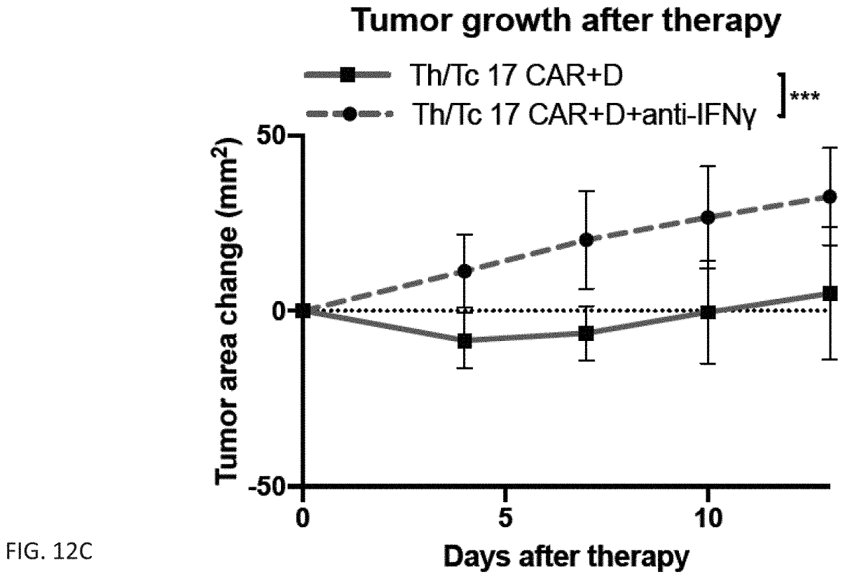

A combination of Tc17 and Th17 cells were necessary for anti-tumor efficacy using DMXAA as Tc17 cells alone were not effective in tumor control (FIG. 10). To evaluate the effect of DMXAA on CD4⁺ T cells, scRNA-seq was performed of CD4⁺ CAR T cells with or without DMXAA treatment (FIG. 11A). Like the findings with CD8⁺ T cells, there was an increase in CAR T cells expressing canonical genes for the Th17 phenotype (RORC) and genes for a cytolytic Th17/Th1 phenotype (TBX21 and IRF4). An increase was also found in the expression of IFNG and chemokine receptor genes found on Th1 cells (CXCR3 and CCR5) (FIGS. 11B-11D). Single cell data was confirmed using flow cytometry, which demonstrated an increase in the number and percentage of CD4⁺ CAR T cells in the presence of DMXAA. This increase was associated with control of tumor growth (FIG. 11F). To further validate the single cell transcriptome data, bulk qPCR array evaluation was performed of the TME. Increased expression of genes associated with the Th1 and Th17 pathways was found in mice receiving Th/Tc17 CAR T cells and DMXAA treatment (FIGS. 12A-12B). Finally, a critical role was demonstrated for IFN-γ expression by demonstrating the loss of activity of Th/Tc17 CAR T cell therapy plus DMXAA in mice that received anti-IFN-γ mAb (FIG. 12C). Thus, DMXAA therapy was associated with a change in the phenotype of Th/Tc17 CAR T cells to a pro-inflammatory dual Th17-Th1/Tc17-Tc1 phenotype, which is critical for tumor control.

CAR T cell Exhaustion Limits In Vivo Efficacy

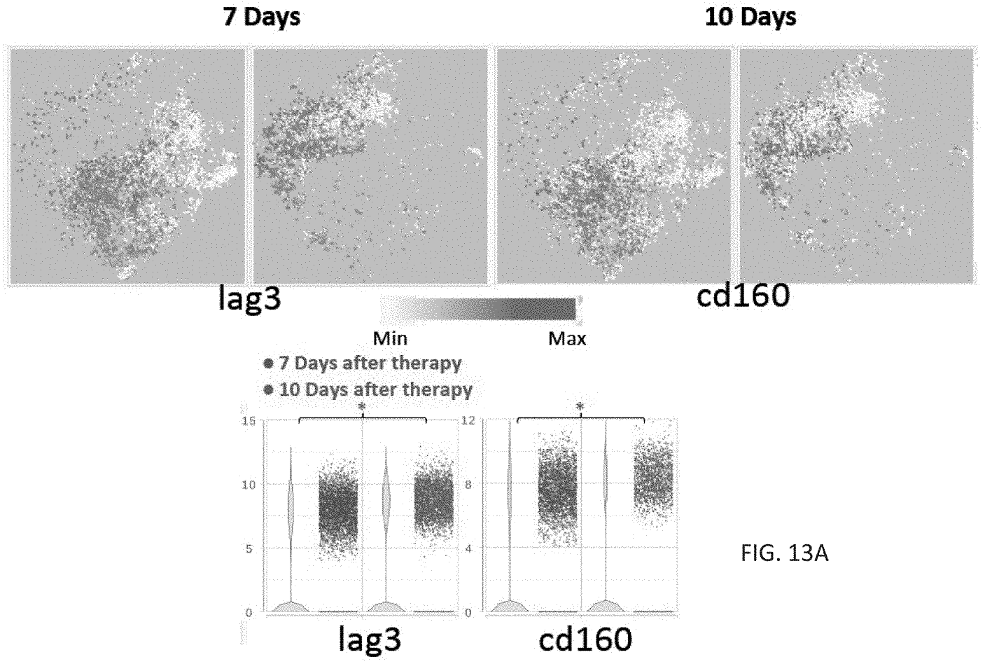

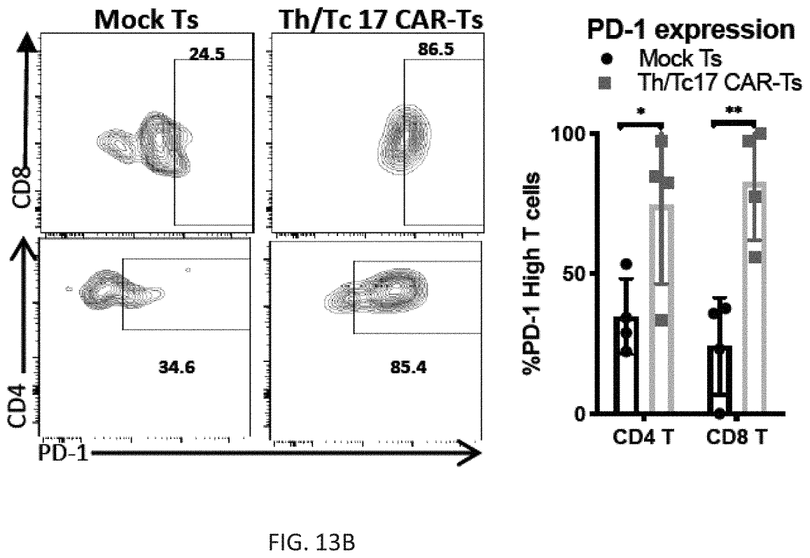

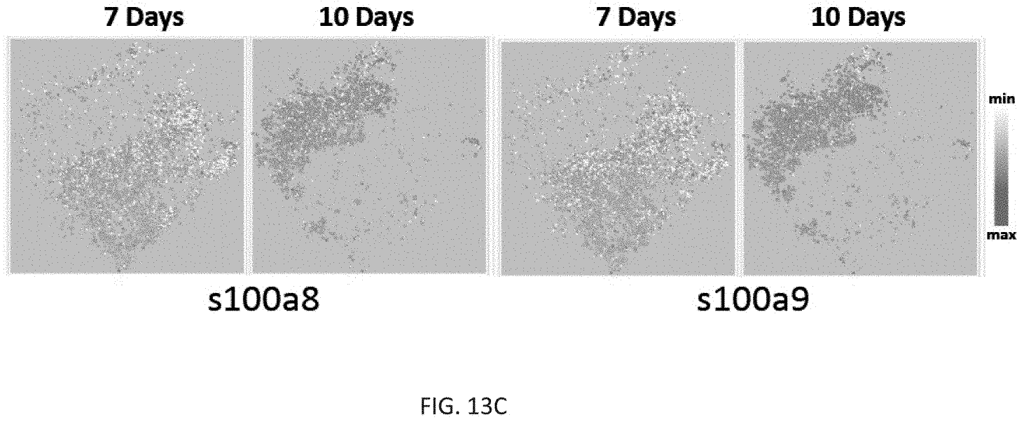

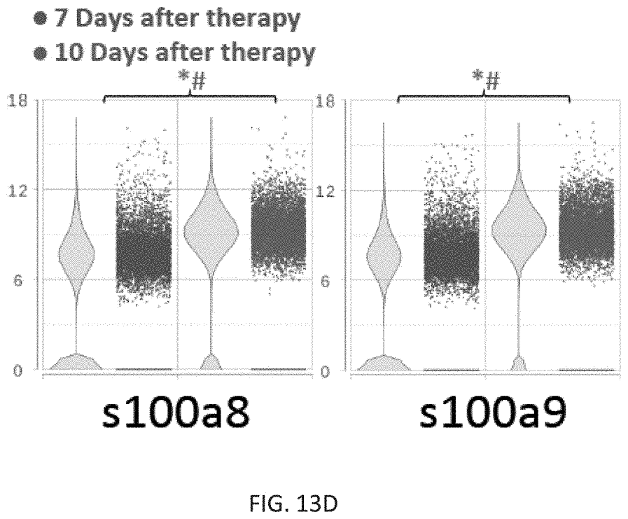

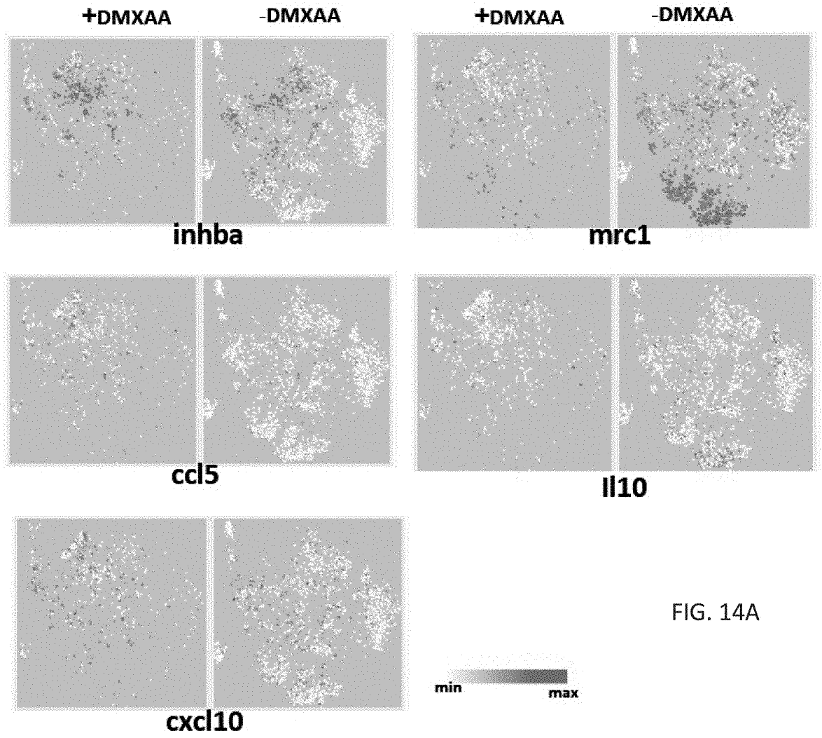

LH28z Th/Tc17 CAR T cell therapy given with DMXAA controlled tumor growth for approximately 10 days but did not eradicate the tumor. To investigate the cause of loss of activity of LH28z Th/Tc17 CAR T cells with DMXAA, scRNA-seq of the TME was compared at days 7 and 10 post CAR T cell therapy. As early as day 7 post LH28z Tc/Th17 CAR T cell infusion, increased expression was found of PDCD1 (PD-1), LAG3, CD160 and TOX by CAR T cells with decreased expression of TCF7 (FIGS. 3G-3I, FIG. 13A). To confirm the single cell data, flow analysis was performed on CAR T cells in the TME. There was a significant increase in PD-1-expressing CD4⁺ and CD8⁺ T cells at day 7 after Tc/Th17 LH28z CAR T cell treatment compared to mock transduced CAR T cells (FIG. 13B). Increased expression of S100A8 and S100A9 was found, which together generate the protein calprotectin, that has been shown to induce T cell apoptosis^{10,11} (FIGS. 13C-13D). Additionally, an increase in CXCL2 was found, which has been associated with the recruitment of suppressive myeloid cells into the TME (FIG. 3J).

Alteration of Myeloid Cells in the TME is Associated with the Function of DMXAA

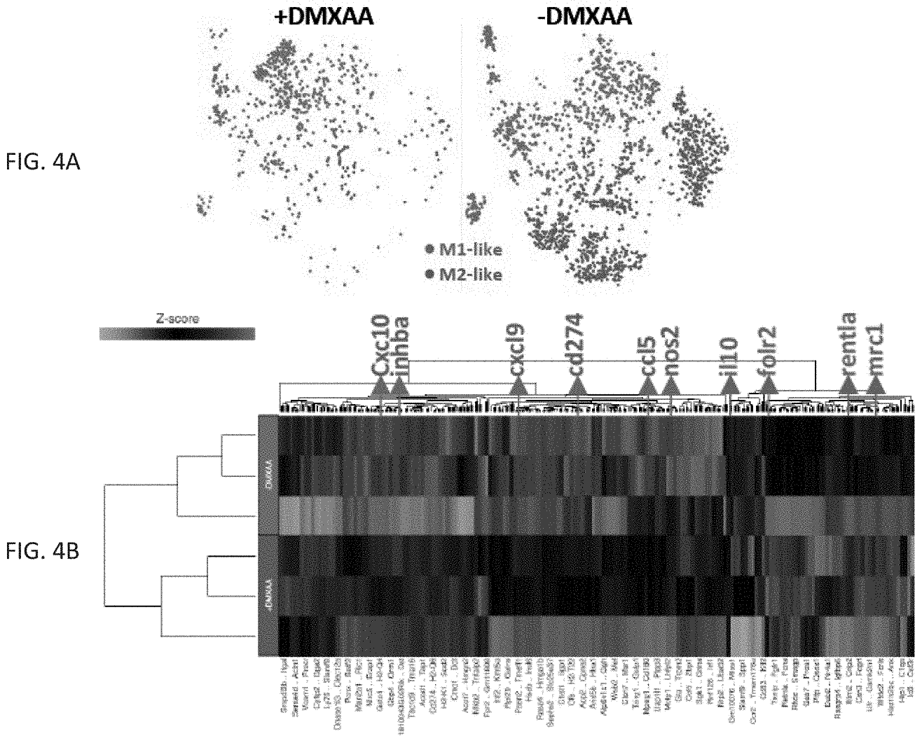

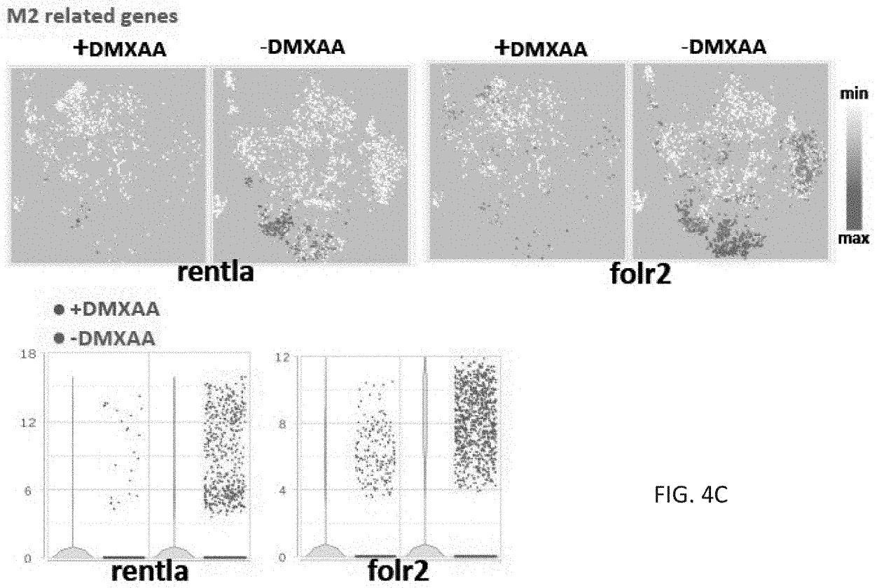

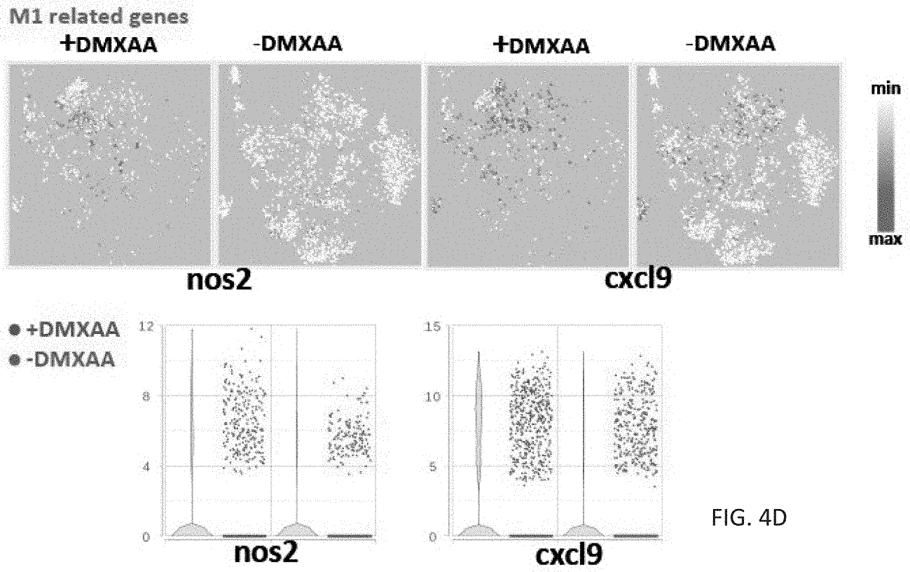

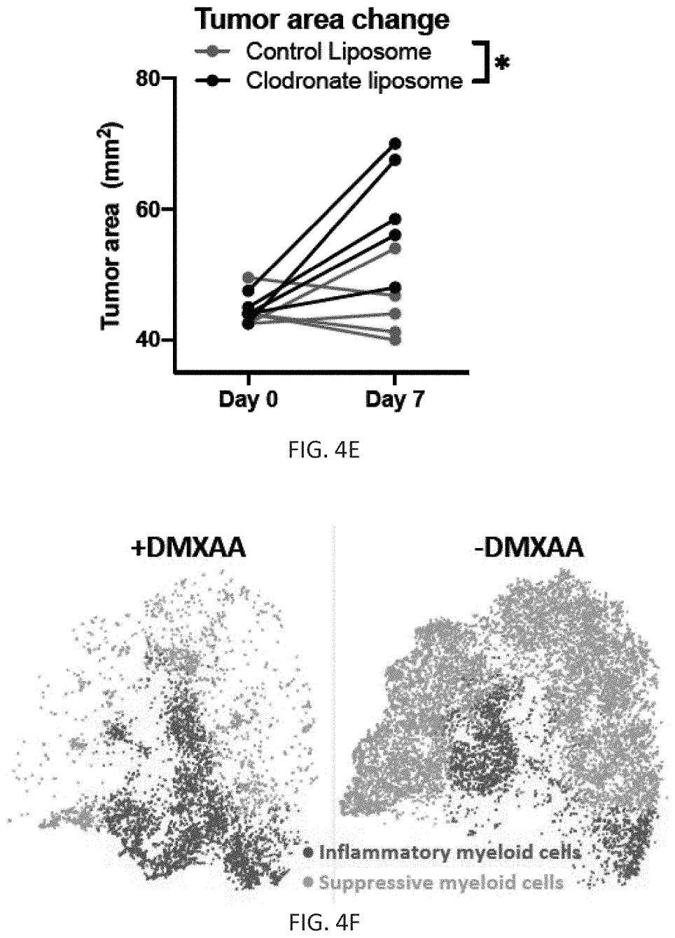

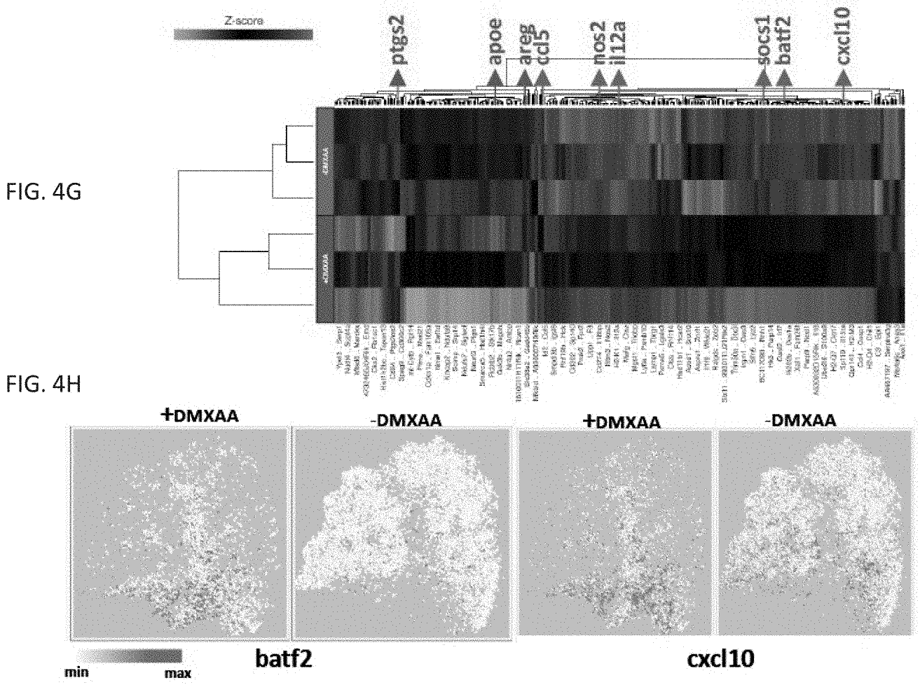

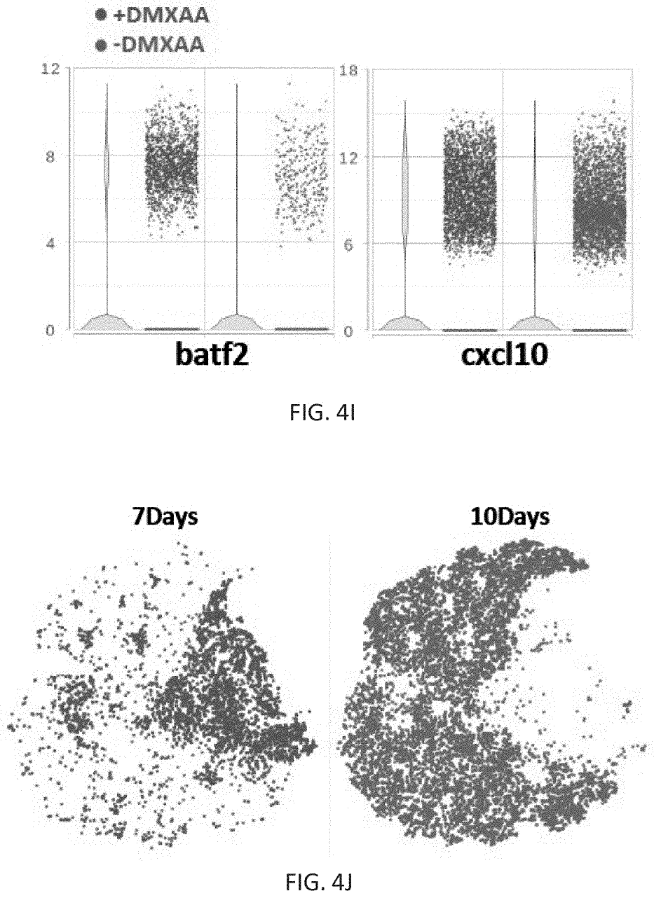

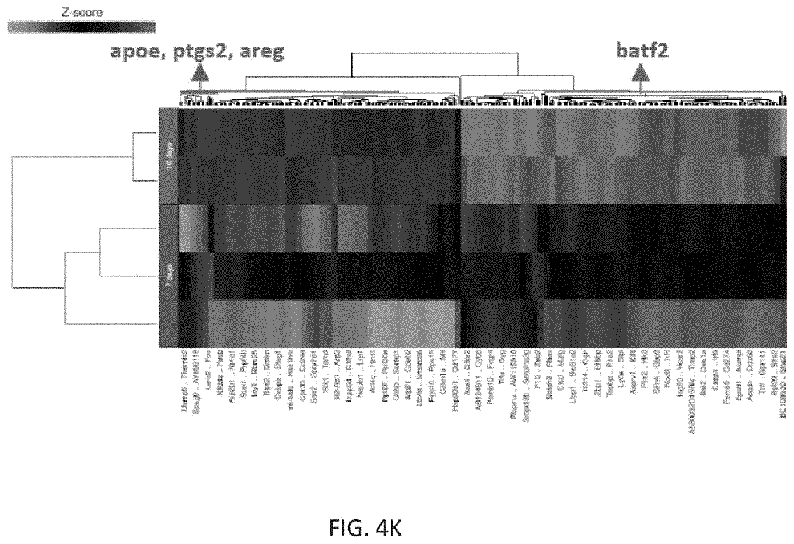

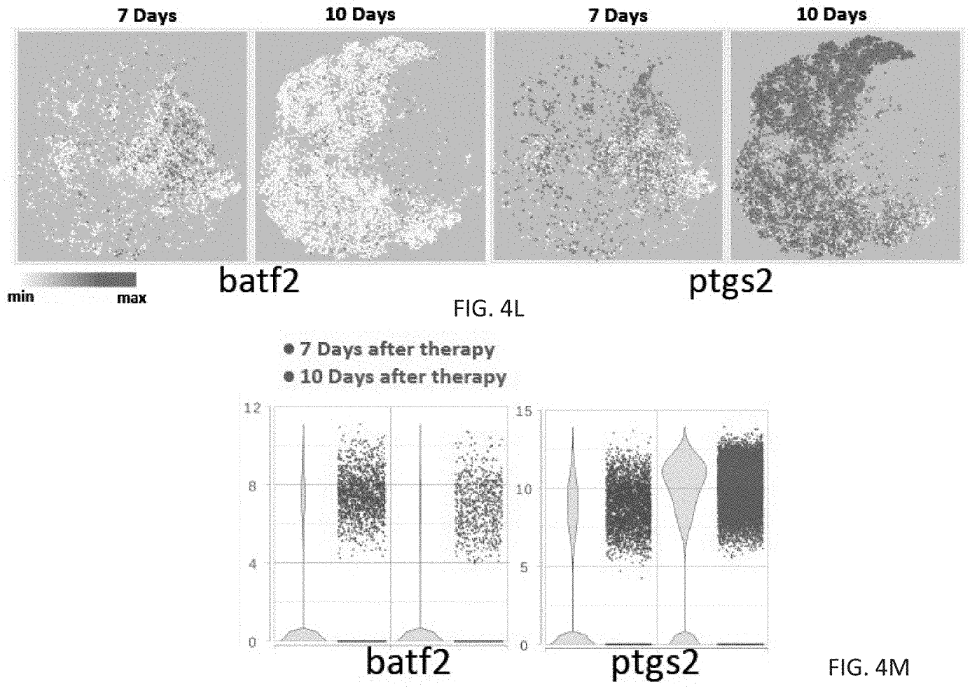

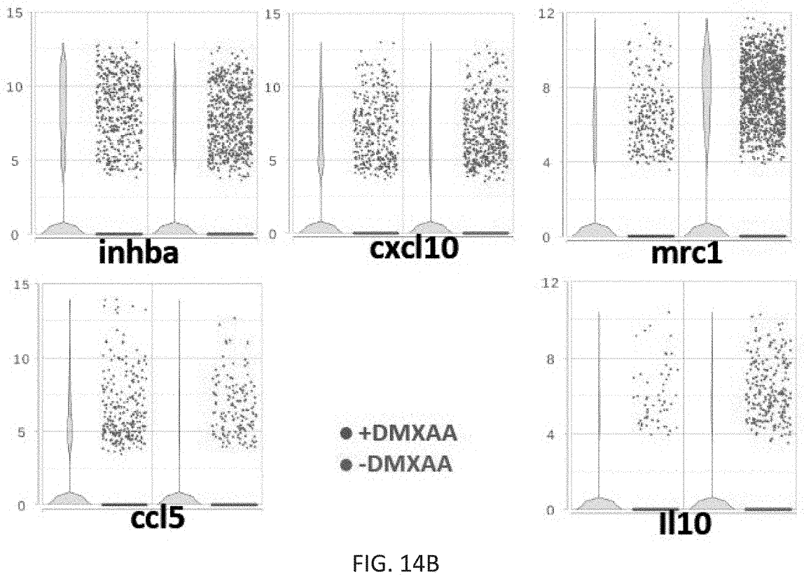

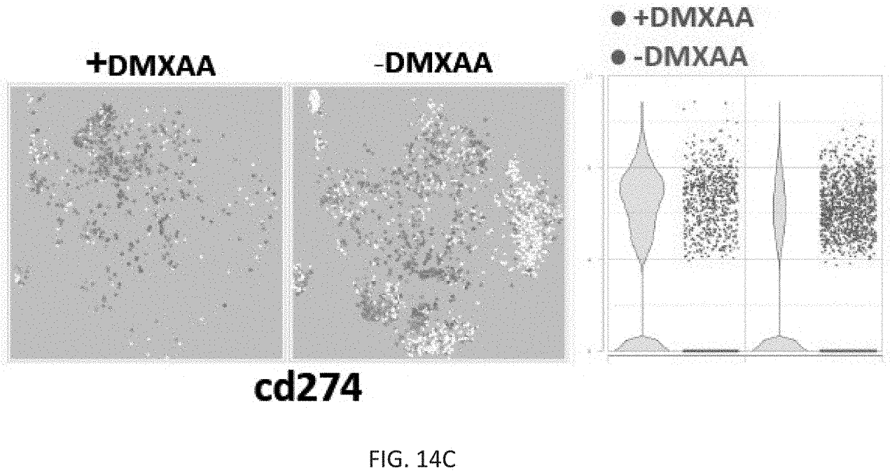

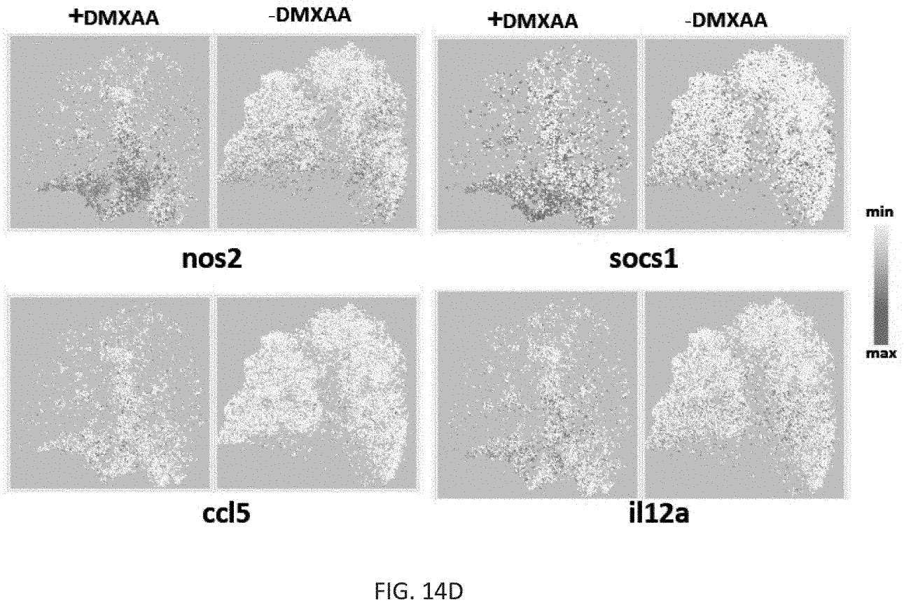

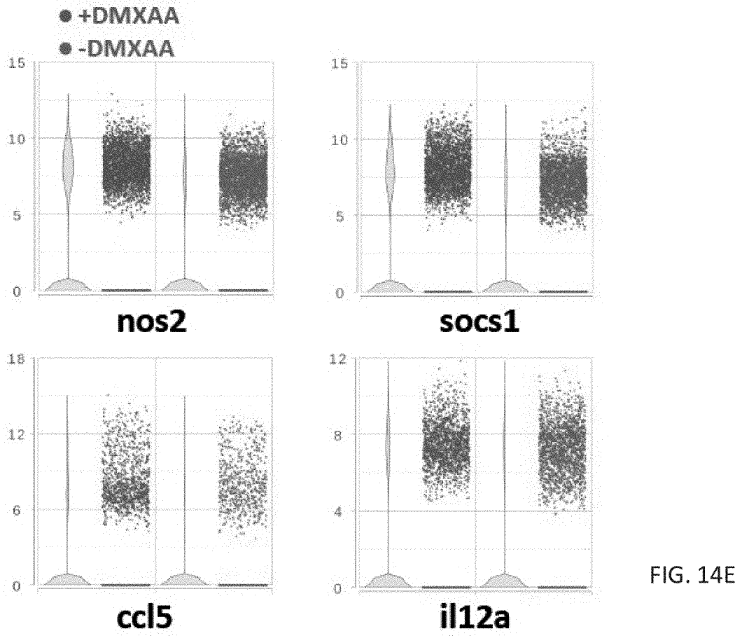

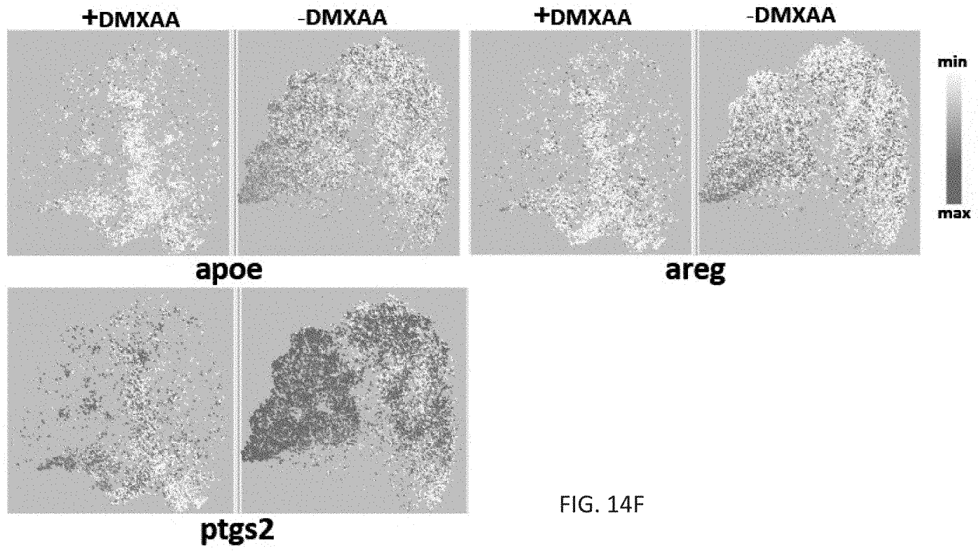

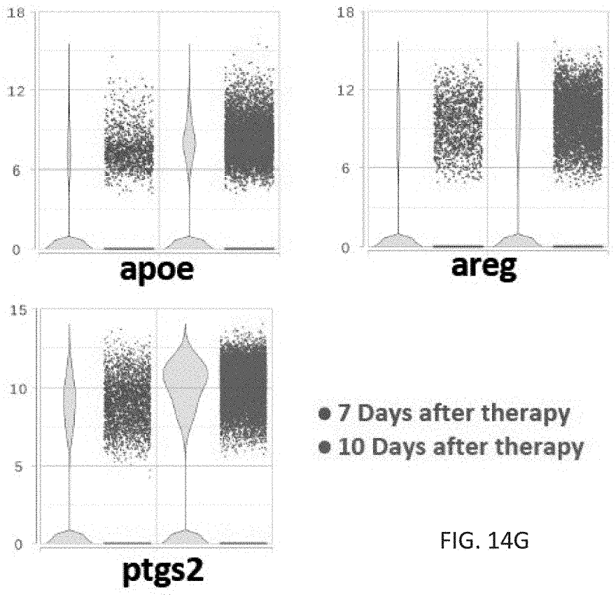

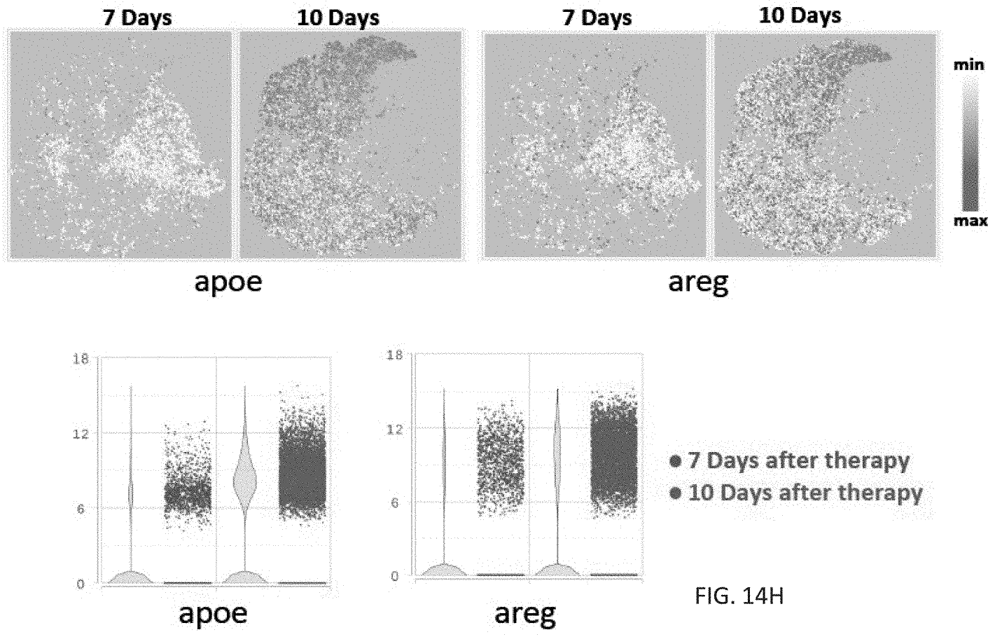

DMXAA can modify the function of innate immune cells^{12,13}. Thus, the effects of the STING agonist on myeloid cells in the TME was evaluated. Significant differences were found in the presence of M1-like and M2-like macrophages, inflammatory-like myeloid cells (iMCs) and myeloid derived suppressor-like cells (MDSC) in mice treated with and without DMXAA (FIGS. 4A-4B). There was a marked decrease in the presence of M2-like macrophages expressing RENTLA, MMRC1, FOLR2, and IL10 in the TME after treatment with DMXAA (FIG. 4C and FIGS. 14A-14B). This was associated with increased expression of genes associated with M1-like macrophages including NOS2 and INHBA. M1-like macrophages had increased expression of chemokines such as CXCL9, CXCL10 and CCL5, which have been shown to recruit Th1/Tc1 T cells by binding to CXCR3 and CCR5-expressing cells (FIG. 4D, and FIGS. 14A-14B). Consistent with the increased expression of IFNG in the presence of DMXAA, macrophages had increased expression of CD274 (PD-L1)¹³ (FIG. 14C). To demonstrate a critical role in the recruitment of CAR T cells to the TME by macrophages, liposome clodronate was used to deplete these cells. Macrophage depletion reversed the efficacy of DMXAA over the first week of Th/Tc17 CAR T cell therapy (FIG. 4E).

A second cluster of myeloid cells was found after treatment with DMXAA with greatly enhanced expression of the transcription factor BATF2, the chemokine ligands CXCL10 and CCL5, and downstream genes related to inflammatory function¹⁵, including SOCS1, NOS2 and IL12A (FIGS. 4F-4I, FIGS. 14D-14E). Additionally, there was reduced expression of APOE, PTGS2 and AREG, and (FIGS. 14F-14G) a gene expression profile consistent with MDSC-like cells. Thus, DMXAA was able to modulate the expression of genes associated with enhanced anti-tumor efficacy and to decrease the presence of pro-tumorigenic myeloid cells.

To characterize the TME immediately prior to tumor growth, myeloid cell subsets were evaluated at day 10 post Th/Tc17 CAR T cell infusion with DMXAA. As shown in FIGS. 4J-4M, there was a significant change in the TME over this four-day period with a marked increase in the presence of MDSC-like cells expressing PTGS2 (Cox2), AREG, and APOE (FIG. 14H). There was a significant decrease in the presence of BATF2-expressing myeloid cells (FIGS. 4L-4M). Thus, the diminished function of LH28z Th/Tc17 CAR T cells plus DMXAA was strongly associated with the re-establishment of an immunosuppressive TME.

### Targeting CAR T Cell Exhaustion and Immature Myeloid Cells Enhanced CAR Efficacy

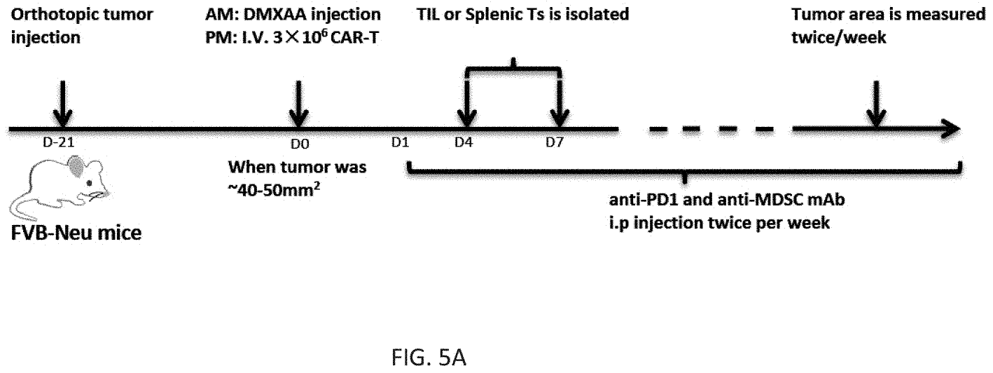

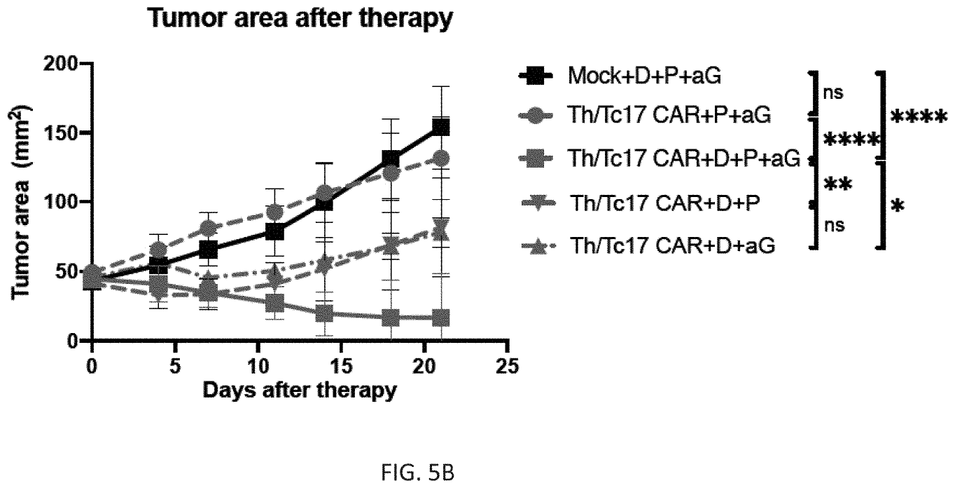

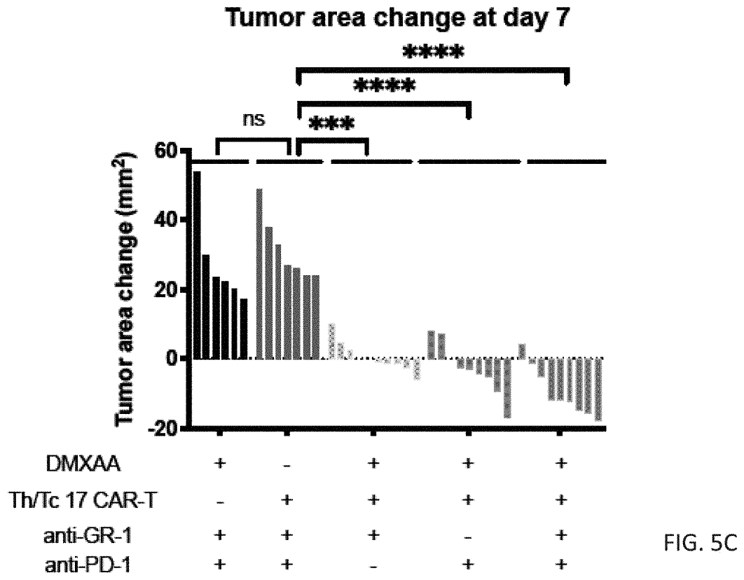

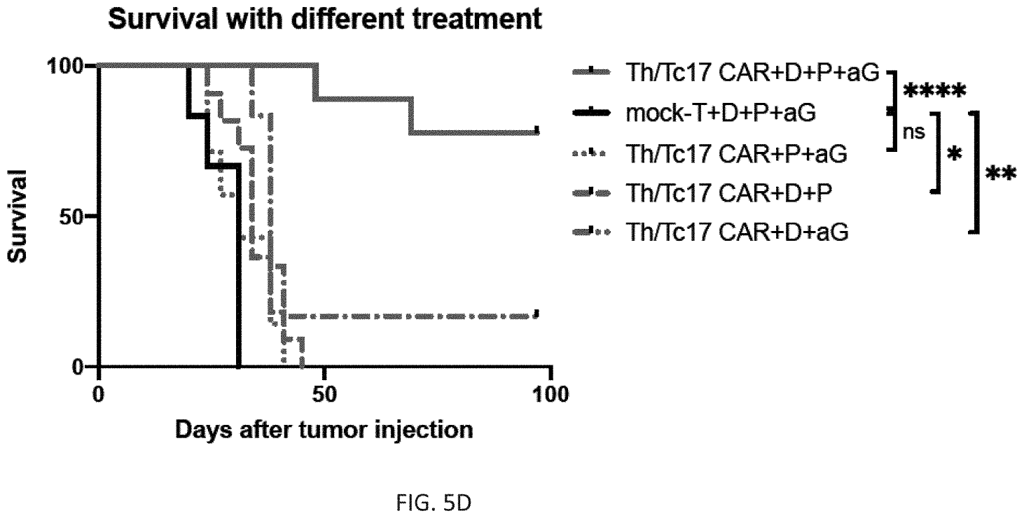

Given the data regarding the expression of PD-1 on CAR T cells and the marked changes in the immunosuppressive nature of the TME, targeting these pathways was evaluated as to whether they could enhance the long-term efficacy of LH28z Th/Tc17 CAR T cells plus DMXAA. To this end, mice were treated with LH28z Th/Tc17 CAR T cells plus DMXAA along with anti-PD-1 and anti-GR-1 mAb therapy (FIG. 5A). This approach led to significantly improved overall survival and control of tumor growth compared to mice that received CAR T cell therapy with DMXAA and either anti-PD-1 or anti-GR-1 mAb therapy (FIGS. 5B-5D). However, substantial anti-tumor activity was found only in mice that received CAR T cells plus DMXAA with either anti-PD-1 or anti-GR-1 mAb therapy (FIGS. 5B-5C). There was no anti-tumor activity in mice that did not receive CAR T cells or DMXAA (FIGS. 5B-5C). Importantly, optimal tumor control required CAR T cell therapy with DMXAA and anti-PD-1 and anti-GR-1 mAbs (FIG. 5D).

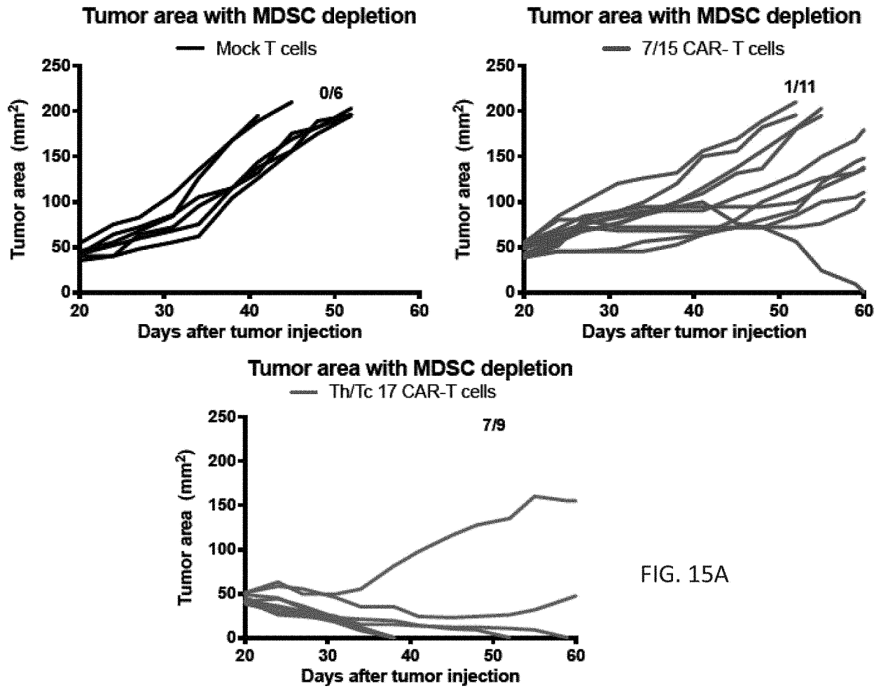

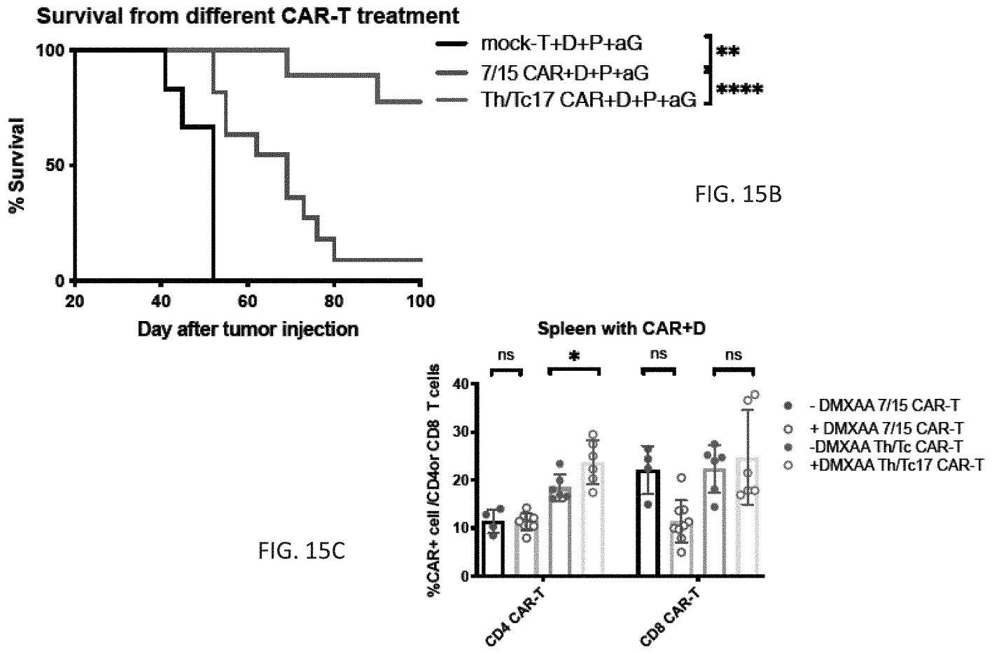

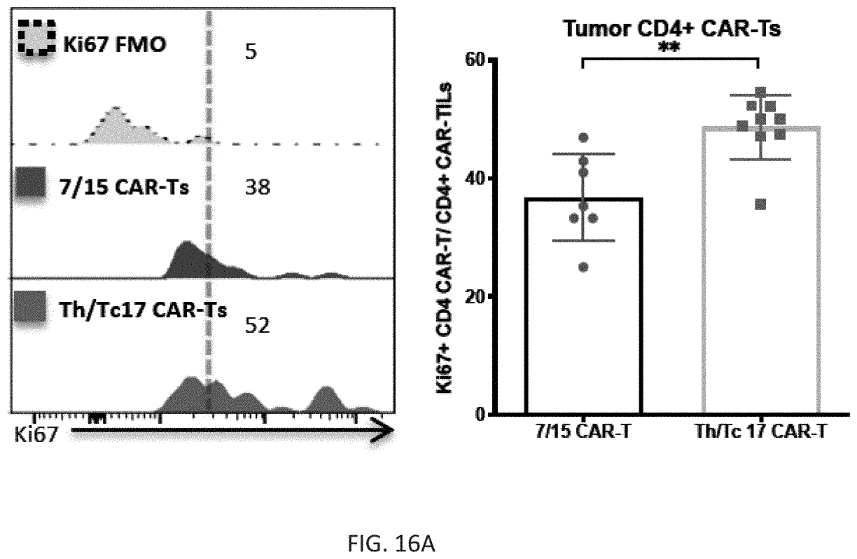

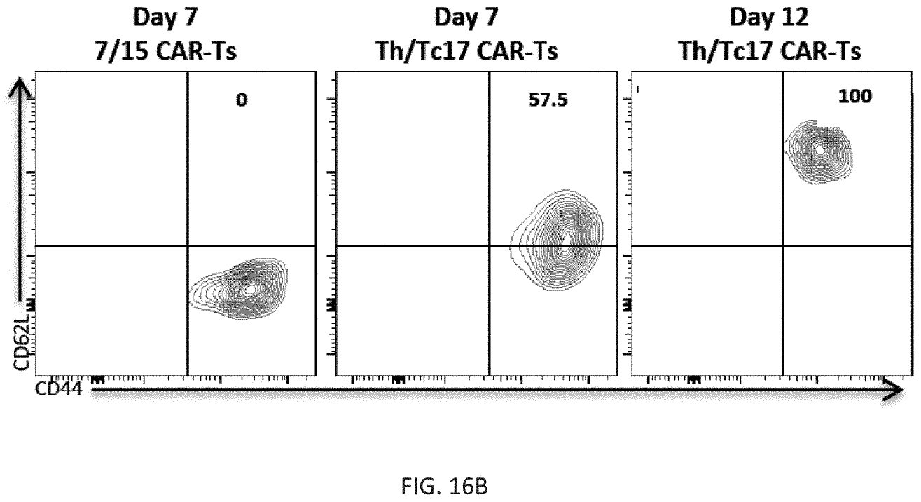

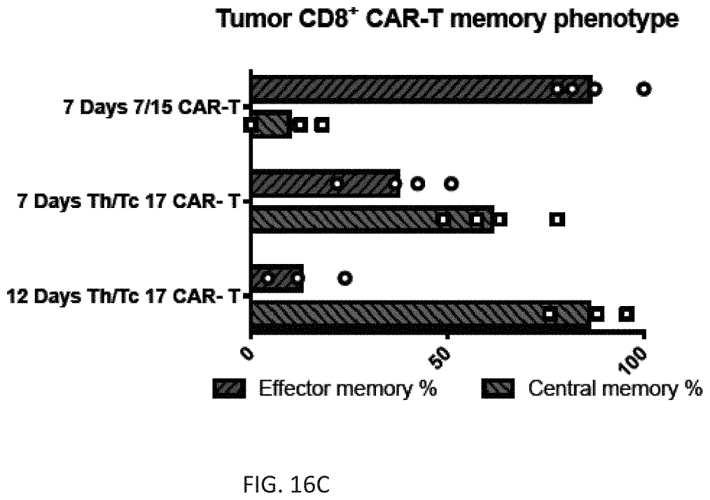

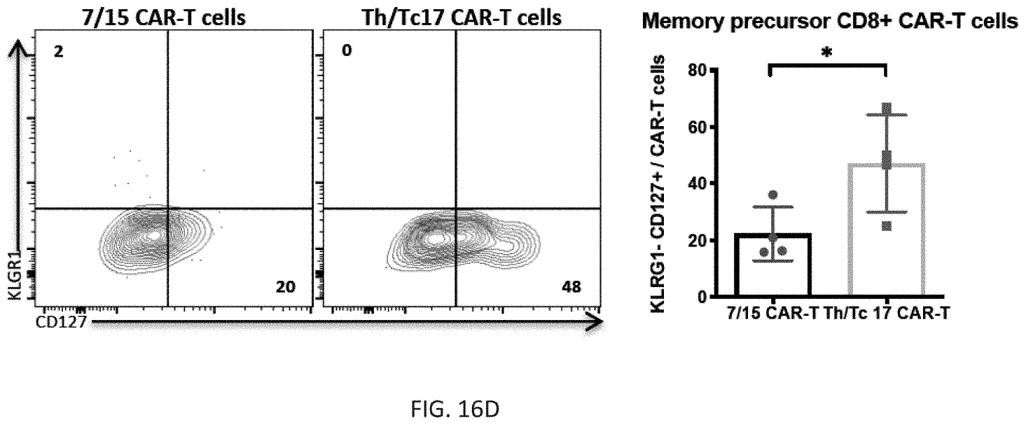

To evaluate whether combination therapy with DMXAA, anti-PD-1 and anti-GR-1 mAbs could also enhance the anti-tumor activity of 7/15 CAR T cells, tumor growth was compared in mice receiving anti-GR-1 and anti-PD-1 mAb with DMXAA and given either 7/15 or Th/Tc17 CAR T cells. Much greater anti-tumor activity was found with the administration of Th/Tc17 CAR T cells compared to 7/15 CAR T cells when combined with DMXX and anti-PD-1 and anti-GR-1 mAbs (FIGS. 15A-15B). Even in the absence of anti-PD-1 and anti-GR-1 therapy, there was an increase in CAR T cells in the TME (FIG. 2A), compared to the spleen (FIG. 15C) in mice receiving Th/Tc17 CAR T cells compared to 7/15 CAR-T cells. There was much greater proliferation of CD4⁺ CAR T cells in mice receiving Th/Tc17 compared to 7/15 CAR T cells (FIG. 16A). For CD8⁺ CAR T cells, a significant increase in the number of CD8⁺ T_{cm} CAR T cells was found from day 7 until day 12 post-infusion in the TME of mice receiving Th/Tc17 as compared to 7/15 CAR T cells, which were predominantly effector memory T cells at day 7 (FIGS. 16B-16C). Differences in the number of effector vs. central memory CAR T cells were supported by the increased number of Th/Tc17 CAR T cells with a precursor effector phenotype (CD127⁺ and KLRG1⁻) (FIG. 16D).

In conclusion, sustained tumor regression caused by Th/Tc17 CAR-T cells was associated with enhanced proliferation and expansion of central memory T cells.

Efficacy Associated with Cytokine-Like Release Syndrome

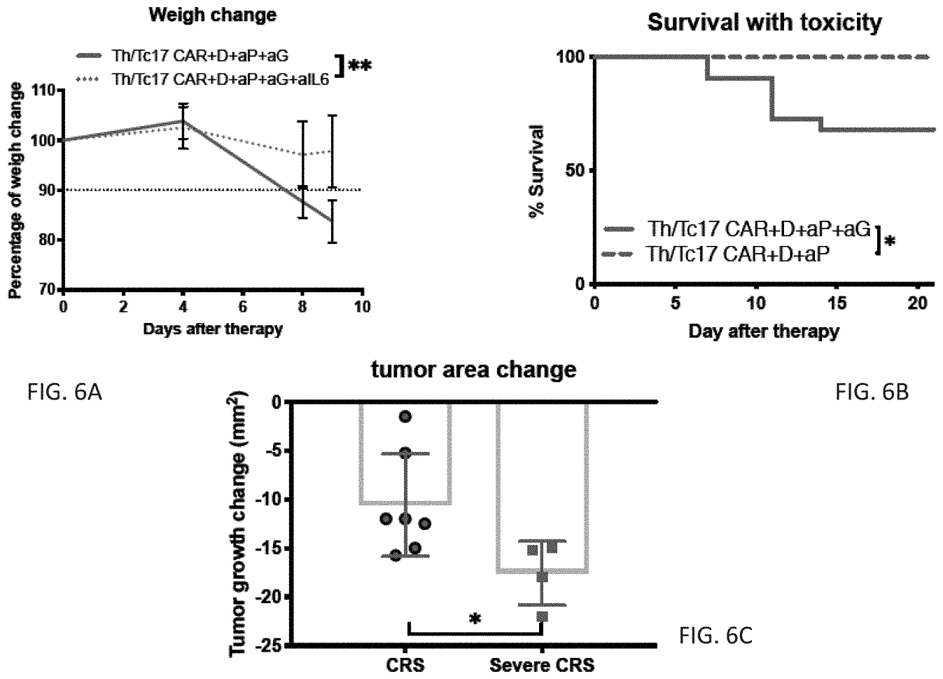

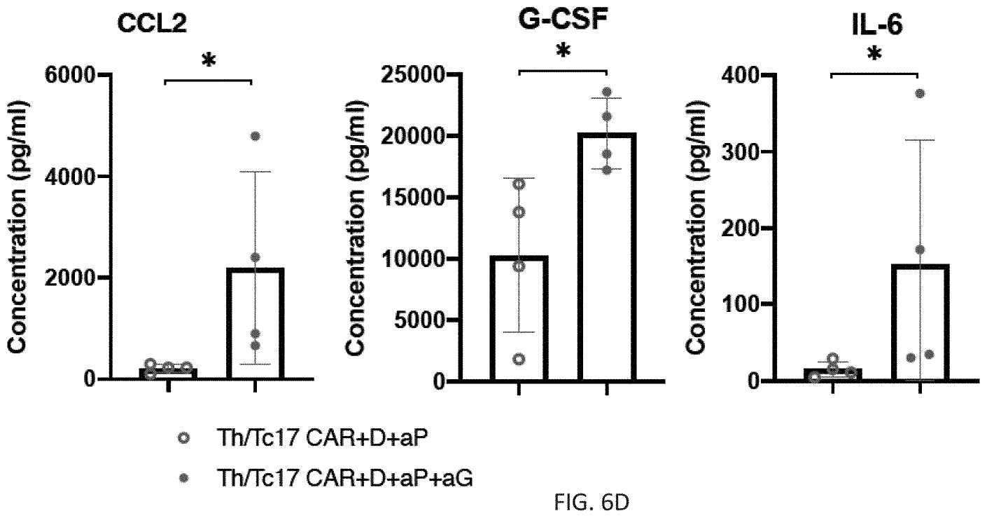

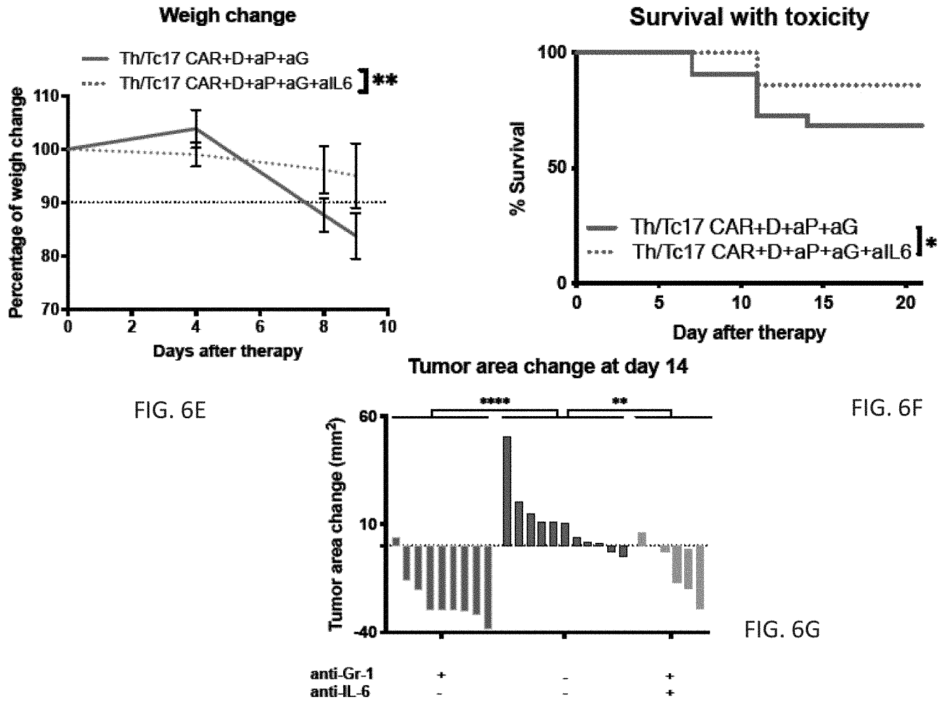

Treatment of mice with Th/Tc17 CAR T cells, DMXAA, and anti-PD-1 and anti-GR-1 mAbs led to significant tumor control with the majority of mice tumor-free 30 days post treatment. However, a small subset of mice that received this combination therapy was found to exhibit significant weight loss and hunching consistent with a cytokine-release like syndrome (FIG. 6A). This was correlated with increased mortality in these mice (FIG. 6B). Treatment efficacy correlated with the development of this syndrome (FIG. 6C). To determine a cause for the increased toxicity in these mice, cytokine levels from the serum of mice receiving Th/Tc17 CAR with DMXAA and anti-PD-1+/−anti-GR-1 mAb treatment was evaluated. Significant increases in CCL2, G-CSF and IL-6 were found in mice given anti-GR-1 mAb compared to mice that did not receive anti-GR-1 treatment (FIG. 6D). Mice receiving Th/Tc17 CAR T cells with DMXAA, anti-PD-1 and anti-GR-1 mAbs, were then treated with an anti-IL-6 antibody, which has demonstrated efficacy in the treatment of CRS in human subjects receiving CAR T cells^{16,17}. Mice were given anti-IL6 antibody at day 5 at the onset of weight loss in animals treated with combination therapy (FIG. 6B). As shown (FIGS. 6E-6G), treatment with anti-IL-6 mAb was associated with improved survival in mice receiving combination therapy.

### Discussion

Although CAR T cells targeting CD19 have been successful in treating patients with B cell malignancies^{1,2}, the clinical activity of CAR T cells in patients with solid tumors has been modest^{5,6}. Using a rational and sequential approach, a combinatorial immunotherapy strategy was implemented based on CAR T cells polarized to a Th/Tc17 phenotype, anti-PD1 mAb, STING agonist DMXAA and anti-GR-1 mAb that leads to tumor eradication in an immunocompetent syngeneic model of breast cancer. The most compelling finding was the ability of DMXAA, given at a site remote of the tumor, to promote CAR T cell recruitment and persistence at the tumor site. It is believed that these data are the first to demonstrate the function of a STING agonist in altering the trafficking properties of adoptive T cellular products. Furthermore, this activity did not require intratumoral injection of the STING agonist.

A critical aspect of effective CAR T cell therapy is the ability of the transferred cells to traffic to tumor sites. CAR T cells expanded ex vivo and infused intravenously in patients home physiologically to secondary lymphoid organs such as the bone marrow and lymph nodes, which explains in part the robust antitumor effects of CAR T cells observed in patients with leukemia, lymphoma and multiple myeloma¹⁸. In contrast, T cells do not constitutively traffic to breast tissue. Th/Tc17 cells have enhanced biodistribution to peripheral tissues as compared to Th1 cells, and this may explain in part our observation that Th/Th17 CAR T cells showed superior persistence in the tumor compared to IL7/15 CAR T cells (FIG. 2 and FIG. 7). To further increase CAR T cell accumulation in solid tumors, engineering processes have been exploited using tumor-associated chemokine gradients or by modifying the stiffness of the extracellular matrix¹⁹⁻²¹. Here, it is reported that the STING agonist DMXAA promoted CAR T cell persistence within the TME. DMXAA treatment led to increased expression of CXCL9 and CXCL10 by myeloid cells within the TME. These chemokines recruit CXCR3-expressing Th/Tc1 T cells. Correlating with this finding, DMXAA treatment was associated with a marked increase in the number of CAR T cells through day 10 post-therapy. Interestingly, these CAR T cells had an altered phenotype as they did not generate IFN-7 prior to infusion but expressed IFNG, TBX21, and CXCR3 post-therapy with DMXAA. T cells expressing IL-17A and IFN-7 have been found in other models to induce significant tissue pathology^{22,23}. The data using anti-IFN-7, which led to a complete loss of the activity of these CAR T cells in vivo, would support a critical role for the conversion of Th/Tc17 CAR T cells to a Th/Tc1 phenotype in their anti-tumor activity in vivo.

The immunocompetent mouse model developed allowed for the assessment of how the myeloid tumor compartment affected the antitumor activity of CAR T cells. It was demonstrated that the persistence of Th/Tc17 CAR T cells in the TME was associated with enhanced expression of genes associated with M1-like macrophages and a marked loss of genes associated with M2-like macrophages and MDSC-like cells. The decrease in BATF2-expressing iMCs was associated with the inability of CAR T cells to control tumor growth. Thus, these data suggest that enhancing CAR T cell trafficking and persistence without altering the immunosuppressive TME is unlikely to lead to significant sustained anti-tumor effects in solid tumors.

Th/Tc17 CAR T cell persistence within the TME, which is promoted by the pro-inflammatory myeloid switch caused by a STING agonist, does not prevent exhaustion of CAR T cells, which requires checkpoint blockade to sustain CAR T cell mediated tumor control. Moreover, a rapid influx of immunosuppressive monocytic MDSCs within the TME was also observed and associated with diminished control of tumor growth. Previous work had shown that CAR T cells specific for CEA were inhibited by liver myeloid derived suppressor cells when given via the portal vein to mice with liver metastasis²⁶. Here, it was found that depletion of MDSC-like cells in combination with CAR T cells, STING agonist and PD-1 blockade allowed sustained regression of the tumor. However, it was observed that the optimal combination also led to the development of rapid cachexia consistent with a cytokine release syndrome. Recent work has highlighted the critical role of myeloid cells in the pathogenesis of cytokine release syndrome²⁷⁻²⁹. The data suggests that MDSCs, and perhaps other immunosuppressive myeloid cells, limit the anti-tumor efficacy of CAR T cells but also attenuate the severity of the inflammatory reaction. Fully licensing CAR T cells by depleting MDSCs exacerbated a cytokine release syndrome, which responded to IL-6 blockade as observed in patients receiving CAR T cells targeting CD19.

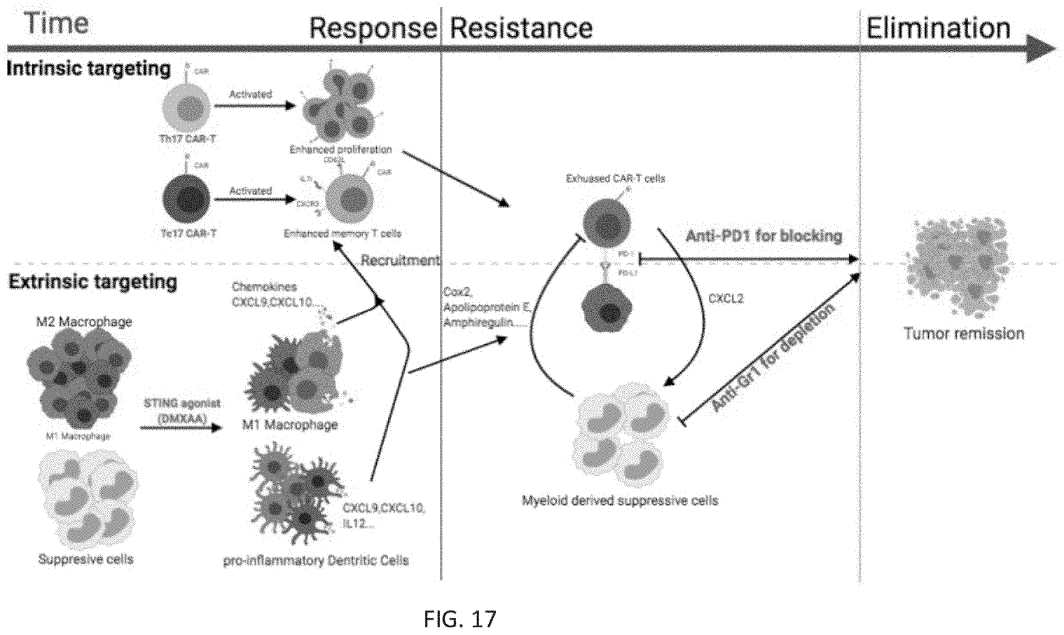

In summary (FIG. 17), it was found that suboptimal trafficking and persistence/expansion of CAR T cells greatly limits their antitumor activity in an orthotopic model of locally advanced breast cancer. The STING agonist, DMXAA, greatly enhanced the trafficking and persistence of CAR T cells especially when Th/Tc17 cells were used to generate CAR T cells. These data suggest a viable strategy for boosting CAR T activity in solid tumors as STING agonists are in clinical trials for the treatment of patients with cancer³⁰, there are multiple ongoing clinical trials using approaches to inhibit MDSCs for patients with malignant disease³¹ and there are clinical trials currently evaluating the combination of CAR T cells with checkpoint blockade.

### Materials and Methods

### Tumor Cell Culture

3T3 cell lines were obtained from the ATCC and cultured in DMEM (Gibco) supplemented with 10% fetal bovine serum (FBS; Gemini), and 1% Pen/Strep (Invitrogen). 3T3-Neu and NT2 tumor have been previously described³⁰. 3T3-Neu cultures were made as described and cultured with the addition of 0.3 μM methotrexate. NT2 (Passage 6) were cultured in RPMI-1640 (Gibco) supplemented with 20% FBS, 2 mM L-glutamine (Invitrogen), 12 mM HEPES (Invitrogen), 0.1 mM NEAA (Invitrogen), 1 mM sodium pyruvate (Invitrogen), 1% Pen/Strep, 50 μM 2-mercaptoethanol (Invitrogen) and 0.2 U/ml Novolin R-insulin (Novo Nordisk). The virus encoding the LH28z CAR was cultured in PLAT-E cells from Cell Biolabs Inc. in DMEM supplemented with 10% FBS and 1% Pen/Strep. All cells used for the generation of the CAR constructs were used within 3 passages in vitro and 0.05% trypsin (Invitrogen) was used for digestion between passages.

### Vector Construct

7/15 and Th/Tc17 CARs contain the scFv of LH28z construct generated from the 7.16.4 antibody. Whole RNA from the 7.16.4 hybridoma was isolated (RNEasy Kit, Qiagen) and made into cDNA (M-MLV Reverse Transcriptase kit, Invitrogen). Antibody heavy and light chains were cloned with IgH or IgL primer mix (iRepertoire Inc.) and sequenced by Sanger sequencing (Genewiz). CAR cDNAs were cloned into a construct containing murine CD8 transmembrane domain and intracellular domain from murine CD28 or 4-1BB and CD3ξ (LH28z, LHBBz). The CAR construct was then cloned into MSGV plasmid and driven by 5′ LTR. The plasmid construct have been previously described³².

### Viral Construct Preparation and Transduction

Plasmids encoding CAR (LH28z) and pCL749 Eco retrovirus packaging vector (Novus) were co-transfected into PLAT-E cells using lipofectamine 2000 (Invitrogen). Supernatant from LH28z-transfected PLAT-E cells was collected after 48 hours and stored at −80° C. For T cell transduction, LH28z or LHBBz or LH28z/LHBBz virus were plated onto a retronectin-coated plate (Takara) and incubated at 32° C. for 2 h followed by the additional of activated T cells. The co-culture was spun at 1200×g for 90 minutes and then stored at 370 C overnight.

### T Cell Activation and Culture

For 7/15 CAR T cells, splenocytes from 8-14 w old female 757 FVB/NJ mice (Jackson Labs) were harvested and activated using plate bound anti-CD3 (145-2C11, Invitrogen) and anti-CD28 mAb (37.51, Invitrogen) and cultured in RPMI-1640 supplemented with 10% FBS, 2 mM L-glutamine, 12 mM HEPES, 0.1 mM NEAA, 1 mM sodium pyruvate, 1% Pen/Strep, 50 μM 2-ME, along with the addition of 1 μg/ml IL-7 and IL-15 (Peprotech). 48 hours later T cells were transduced with retrovirus and cultured for an additional 3 days with IL-7 and IL-15. For Th/Tc17 CAR T cells, naïve CD4+ and CD8+ T cells were isolated from the spleen of 8-14 week old female FVB/NJ mice (Jackson Labs) using murine naive CD4/CD8 T Cell Isolation Kit (Miltenyi) according to the manufacturer's instructions. T cells were activated using plate bound anti-CD3 and anti-CD28 mAb (Invitrogen). For the first week, the culture was maintained as described above with the addition of TGF-β1 (2 ng/ml), IL-1β (10 ng/ml), TNF (20 ng/ml), IL-6 (30 ng/ml) (Peprotech), anti-IFNγ (R4-6A2, 10 μg/ml, Bioxcell) and anti-IL-2 (JES6-5H4, 10 μg/ml, Bioxcell) (Th17 cytokine cocktail). Two days post-activation, T cells were transduced with retrovirus and kept in culture with Th17 cytokine cocktail for the rest of the week. During the second week, cultured T cells were re-activated using plate bound anti-CD3 and anti-CD28 mAb and maintained in Th17 cytokine cocktail plus IL-23 (15 ng/ml) (Peprotech).

### Murine Tumor Model

8-12 week old female FVB-Neu mice (Jackson Labs) were used under a protocol approved by University of North Carolina Institutional Animal Care and Use Committee. For tumor inoculation, mice were intradermally injected with 5×10⁴ NT2 tumor cells into an axillary mammary gland. 21 days later (day 0), 1.5×10⁶ CD4+ and 1.5×10⁶ CD8+ CAR T cells were injected intravenously. Tumors were measured twice weekly with calipers and the tumor area calculated as length×width. The change of tumor area was determined by subtracting the tumor area before CAR T injection with the value on the day of the measurement. Per institutional guidelines, tumor-bearing mice were sacrificed when the tumor area reached 200 mm² or loss of more than 20% of total body weight. For tumor-bearing animals receiving combination therapy, animals underwent lymphodepletion with 5 Gy of whole body irradiation (X-RAD 320, Precision X-Ray) one day prior to CAR T injection. In the morning of day 0 mice were injected with 500 μg of DMXAA (Tocris) distal to the site of tumor growth with CAR T cells injected in the afternoon. Starting 1 day after CAR T infusion, anti-PD-1 (J43, 200 μg/mouse), anti-Gr-1 (RB6-8C5, 300 ag/mouse), and anti-IFN-7 (R4-6A2, 250 μg/mouse) mAb were injected intraperitoneally into respective recipients.

### In Vivo Tumor Infiltrating Immune Cells Isolation

At indicated time points, tumors were bluntly resected and cut into small pieces and processed into a single-cell suspension using gentleMACS™ Dissociator (Miltenyi). Lymphocyte enrichment was achieved by isolating cells at the interface of 44% Percoll (Sigma-Aldrich) in media and Lympholyte-M (Cedarlane). Isolated immune cells were stained as described below. The lymphocyte fraction was not enriched in experiments where absolute cell number was determined.

### In Vivo Tumor Infiltrating Immune Cells Isolation for Single Cell RNA Sequencing Analysis

At day 7 or day 10 after CAR-T therapy, tumors were bluntly resected and cut into small pieces and digested using Tumor Dissociation Kit, mouse (Miltenyi) in gentleMACS™ Dissociator (Miltenyi) according to manufacturer's instruction. After digestion, dead and dying cells were magnetically separated using a Dead Cell Removal Kit (Miltenyi), after which immune cells were magnetically enriched using CD45 (TIL) MicroBeads (Miltenyi). Single cells were partitioned using a Chromium Controller (10× Genomics), and gene expression sequencing libraries were generated using Chromium Single Cell 3′ Library & Gel Bead Kit v3 (10× Genomics). The libraries were pooled and sequenced using an Illumina NovaSeq 6000 (Illumina). Raw base call files were demultiplexed with Cell Ranger 3.1.0 (10× Genomics) and bcl2fastq Conversion Software v2.20.0 (Illumina).

### Single Cell RNA Sequencing Result Analysis

Single cell results from Cell Ranger output was analyzed using Partek flow workstation (available at partek.com/application-page/single-cell-gene-expression/). In detail, single cell result from each sample of Cell Ranger output was filtered using single cell (QA/QC) filter to exclude doublets/multiplets, cells expressing genes <30 genes per cell and dead cells. Processed results were normalized using recommended method, including log normalization of gene expression and scaled to count per million. Following results were filtered out by feature (gene) that were not expressed in 99% of samples. Then the replicate samples were pooled and clustered with an unsupervised method based on PCA using the first 16 principle component. Clustered plots were visualized by t-SNE plot, where cells were clustered using shared nearest neighbor (SNN). Populations was determined by expression of key markers, including Cd3e (T cells), Adgre1 (macrophage) and Itgam and/or Itgax (myeloid cells), and T cells were further clustered by expression of Cd8 and Cd44 (activated CD8 T cells), lack of Cd44 (naive T cells), Cd4 Cd44 (activated CD4 T cells) and Cd4 Foxp3 (T_{reg}). For comparison, samples from each treatment group was pooled by the mean expression to simulate bulk RNA-seq, and compared using GSA analysis directly. Gene hits that reached P<0.05 with more than 2 or less than −2 fold expression difference was reported and shown as a heat map for pooled samples (macrophage and myeloid cells) or Q<0.05 with 2 or −2 fold expression difference for non-pooled samples (T cells). Further detailed analysis steps can be found on Partek flow page (available at documentation.partek.com/pages/viewpage.action?pageId=12943422) and parameter setting was set as default unless indicated.

### Flow Cytometry

Anti-Neu CAR T cells were detected using recombinant Neu/Her2.Fc fusion protein conjugated with PE (Neu-PE) (Creative Biomart). Antibodies used in in vitro assays included anti-CD45-FITC/e450 (30-F11, Invitrogen), anti-IFN-γ-APC (XMG1.2, Invitrogen), anti-TNF-PE-Cy7 (MP6-XT22, Invitrogen) and anti-IL-17A-PE (eBio17B7, Invitrogen). Tumor-infiltrating immune cells were detected and analyzed with anti-CD45-FITC, anti-CD3-e450 (17A2, Invitrogen), anti-CD4-BV510 (GKL.5, Biolegend), anti-CD8-APC-Cy7 (53-6.7 BD Biosciences), anti-CD44-PerCP5.5 (IM7, Invitrogen), anti-CD62L-APC (MEL-14, Invitrogen), anti-CD11b-BV711 (M1/70, Invitrogen), anti-F4/80-BV605 (BM8, Invitrogen), anti-Gr-1-Percp5.5 (RB6-8C5, Invitrogen), anti-PD-1-PE-Cy7 (J43, Invitrogen), anti-CD127-BV421 (A7R34, Biolegend), anti-KLGR1-BV605 (2F1, BD Bioscience). For in vivo intracellular protein staining, anti-Foxp3-PerCP5.5 (FJK-16S. Invitrogen), anti-Ki67-APC (SOLA15, Invitrogen) were used. Dead cells were excluded with Fixable Viability Stain 700 (BD Biosciences) and Fc receptors were blocked with Fc block (BD Biosciences). Cell number was determined using Count Bright Beads (Invitrogen). Data were collected using a BD LSR-Fortessa or BD Canto cytometer (BD Biosciences). Data were analyzed with FlowJo software (Treestar).

### Flow Cytometry-Based T Cell Cytolytic Assay

For co-culture killing assays, 10⁶ NT2 tumor cells were labeled with 5 μM CFSE (Invitrogen) and plated in culture media for 24 hours. CAR-T, mock transfected T cells or no T cells were added the next day at the indicated ratios and co-cultured in T cell media without cytokines for three days. Supernatant and trypsinized cells were collected and stained with 7-AAD (BD Biosciences) and anti-CD45 (30-F11, Invitrogen). Killing was measured by flow cytometry after gating on CFSE⁺, 7-AAD⁻ and CD45⁻ tumor cells and normalized to groups without T cell addition. For killing curve, NT2 tumor cells were labeled with CFSE and plated at 10⁵ cells/well in a 48 well plate. CAR T cells, mock T cells or no T cells at the indicated ratios were added 12 hours later. Co-cultures were maintained in T cell media without cytokine for 18 hours and cell killing was measured as described above. Percent tumor lysis was calculated by subtracting the number of live tumor cells from 100% and dividing by the total number of live tumor cells in the group with no T cells added.

### Cytokine Release Assay

5×10⁵ 3T3 or 3T3-Neu cells were treated with trypsin and processed into a single cell suspension prior to culture with CAR T or mock T cells at indicated ratios in T cell media without additional cytokine. 5 μg/ml of Brefeldin A (BD Biosciences) was added after the first hour co-culture. Cells were stimulated for 5 hours and then pelleted for extracellular staining with anti-CD45, followed by fixation and permeabilization using Fix/Perm Kit (BD Biosciences) according to the manufacturer's instructions. Anti-IFN-7, anti-TNF and anti-IL-17A were used for intracellular cytokine staining and detected by flow cytometry.

### Real-Time PCR Array

Whole tumor RNA was isolated using the RNEasy Kit (Qiagen) and reverse transcribed using RT2 First Strand Kit (Qiagen). RT-qPCR was performed according to the manufacturer's instruction with the RT² Profiler™ PCR Array Mouse Th17 Response (PAMM-073ZE-4, Qiagen) and performed on the QuantStudio 6K (Applied Biosystems Inc.). Ct values were determined by ABI software and data analysis was performed using the web-based RT2 Profiler PCR Array Data Analysis version 3.5 (Qiagen). Fold change was normalized to a housekeeping gene (Gusb).

### REFERENCES

1. Neelapu, S. S. et al. Axicabtagene Ciloleucel CAR T-Cell Therapy in Refractory Large B-Cell Lymphoma. The New England journal of medicine 377, 2531-2544, doi:10.1056/NEJMoa1707447 (2017).
2. Maude, S. L. et al. Tisagenlecleucel in Children and Young Adults with B-Cell Lymphoblastic Leukemia. The New England journal of medicine 378, 439-448, doi:10.1056/NEJMoa1709866 (2018).
3. Gorochov, G., Lustgarten, J., Waks, T., Gross, G. & Eshhar, Z. Functional assembly of chimeric T-cell receptor chains. Int J Cancer Suppl 7, 53-57 (1992).
4. Gross, G. & Eshhar, Z. Endowing T cells with antibody specificity using chimeric T cell receptors. FASEB J 6, 3370-3378 (1992).
5. Beatty, G. L. et al. Activity of Mesothelin-Specific Chimeric Antigen Receptor T Cells Against Pancreatic Carcinoma Metastases in a Phase 1 Trial. Gastroenterology 155, 29-32, doi:10.1053/j.gastro.2018.03.029 (2018).
6. Tchou, J. et al. Safety and Efficacy of Intratumoral Injections of Chimeric Antigen
Receptor (CAR) T Cells in Metastatic Breast Cancer. Cancer immunology research 5, 1152-1161, doi:10.1158/2326-6066.CIR-17-0189 (2017).
7. Kryczek, I., Zhao, E., Liu, Y., Wang, Y., Vatan, L., Szeliga, W., . . . Zou, W. (2011).
Human TH17 cells are long-lived effector memory cells. Science translational medicine, 3(104), 104ra100. doi:10.1126/scitranslmed.3002949
8. Muranski, P., Borman, Z. A., Kerkar, S. P., Klebanoff, C. A., Ji, Y., Sanchez-Perez, L., . . . & Roychoudhuri, R. (2011). Th17 cells are long lived and retain a stem cell-like molecular signature. Immunity, 35(6), 972-985.
9. Sen, T., Rodriguez, B. L., Chen, L., Della Corte, C. M., Morikawa, N., Fujimoto, J., . . . & Fan, Y. (2019). Targeting DNA damage response promotes antitumor immunity through STING-mediated T-cell activation in small cell lung cancer. Cancer discovery, 9(5), 646-661.
10. Ghavami, S., Eshragi, M., Ande, S. R., Chazin, W. J., Klonisch, T., Halayko, A. J., . . . & Los, M. (2010). S100A8/A9 induces autophagy and apoptosis via ROS-mediated cross-talk between mitochondria and lysosomes that involves BNIP3. Cell research, 20(3), 314-331.
11. Wang, S., Song, R., Wang, Z., Jing, Z., Wang, S., & Ma, J. (2018). S100A8/A9 in Inflammation. Frontiers in immunology, 9, 1298.
12. Corrales, L., McWhirter, S. M., Dubensky, T. W., & Gajewski, T. F. (2016). The host STING pathway at the interface of cancer and immunity. The Journal of clinical investigation, 126(7), 2404-2411.
13. Woo, S. R., Fuertes, M. B., Corrales, L., Spranger, S., Furdyna, M. J., Leung, M. Y., . . . & Alegre, M. L. (2014). STING-dependent cytosolic DNA sensing mediates innate immune recognition of immunogenic tumors. Immunity, 41(5), 830-842.
14. Garcia-Diaz, A., Shin, D. S., Moreno, B. H., Saco, J., Escuin-Ordinas, H., Rodriguez, G. A., . . . & Parisi, G. (2017). Interferon receptor signaling pathways regulating PD-L1 and PD-L2 expression. Cell reports, 19(6), 1189-1201.
15. Kanemaru, H., Yamane, F., Fukushima, K., Matsuki, T., Kawasaki, T., Ebina, I., . . . & Satoh, T. (2017). Antitumor effect of Batf2 through IL-12 p40 up-regulation in tumor-associated macrophages. Proceedings of the National Academy of Sciences, 114(35), E7331-E7340.
16. Li, J., Piskol, R., Ybarra, R., Chen, Y. J. J., Li, J., Slaga, D., . . . & Junttila, M. R. (2019). CD3 bispecific antibody-induced cytokine release is dispensable for cytotoxic T cell activity. Science translational medicine, 11(508), eaax8861.
17. Giavridis, T., van der Stegen, S. J., Eyquem, J., Hamieh, M., Piersigilli, A., & Sadelain, M. (2018). CAR T cell-induced cytokine release syndrome is mediated by macrophages and abated by IL-1 blockade. Nature medicine, 24(6), 731-738.
18. Hunter, M. C., Teijeira, A., & Halin, C. (2016). T Cell Trafficking through Lymphatic Vessels. Frontiers in immunology, 7, 613. doi:10.3389/fimmu.2016.00613
19. Adachi, K., Kano, Y., Nagai, T., Okuyama, N., Sakoda, Y., & Tamada, K. (2018). IL-7 and CCL19 expression in CAR T cells improves immune cell infiltration and CAR T cell survival in the tumor. Nature biotechnology, 36(4), 346.
20. Moon, E. K., Carpenito, C., Sun, J., Wang, L. C. S., Kapoor, V., Predina, J., . . . & Albelda, S. M. (2011). Expression of a functional CCR2 receptor enhances tumor localization and tumor eradication by retargeted human T cells expressing a mesothelin-specific chimeric antibody receptor. Clinical cancer research, 17(14), 4719-4730.
21. Caruana, I., Savoldo, B., Hoyos, V., Weber, G., Liu, H., Kim, E. S., . . . & Dotti, G. (2015). Heparanase promotes tumor infiltration and antitumor activity of CAR-redirected T lymphocytes. Nature medicine, 21(5), 524.
22. Buonocore, S., Ahern, P. P., Uhlig, H. H., Ivanov, I. I., Littman, D. R., Maloy, K. J., & Powrie, F. (2010). Innate lymphoid cells drive interleukin-23-dependent innate intestinal pathology. Nature, 464(7293), 1371.
23. Hirota, K., Duarte, J. H., Veldhoen, M., Hornsby, E., Li, Y., Cua, D. J., . . . & Garefalaki, A. (2011). Fate mapping of IL-17-producing T cells in inflammatory responses. Nature immunology, 12(3), 255.
24. De La Rochere, P., Guil-Luna, S., Decaudin, D., Azar, G., Sidhu, S. S., & Piaggio, E. (2018). Humanized mice for the study of immuno-oncology. Trends in immunology, 39(9), 748-763.
25. Newick, K., O'Brien, S., Moon, E., & Albelda, S. M. (2017). CAR T cell therapy for solid tumors. Annual review of medicine, 68, 139-152.
26. Burga, R. A., Thorn, M., Point, G. R., Guha, P., Nguyen, C. T., Licata, L. A., . . . & Katz, S. C. (2015). Liver myeloid-derived suppressor cells expand in response to liver metastases in mice and inhibit the anti-tumor efficacy of anti-CEA CART. Cancer Immunology, Immunotherapy, 64(7), 817-829.
27. Staedtke, V., Bai, R. Y., Kim, K., Darvas, M., Davila, M. L., Riggins, G. J., . . . & Zhou, S. (2018). Disruption of a self-amplifying catecholamine loop reduces cytokine release syndrome. Nature, 564(7735), 273.
28. Sterner, R. M., Sakemura, R., Cox, M. J., Yang, N., Khadka, R. H., Forsman, C. L., . . . & Schick, K. J. (2019). GM-CSF inhibition reduces cytokine release syndrome and neuroinflammation but enhances CAR T cell function in xenografts. Blood, 133(7), 697-709.
29. Norelli, M., Camisa, B., Barbiera, G., Falcone, L., Purevdorj, A., Genua, M., . . . & Traversari, C. (2018). Monocyte-derived IL-1 and IL-6 are differentially required for cytokine-release syndrome and neurotoxicity due to CAR T cells. Nature medicine, 24(6), 739.
30. Woo, S. R., Fuertes, M. B., Corrales, L., Spranger, S., Furdyna, M. J., Leung, M. Y., . . . & Alegre, M. L. (2014). STING-dependent cytosolic DNA sensing mediates innate immune recognition of immunogenic tumors. Immunity, 41(5), 830-842.
31. Fleming, V., Hu, X., Weber, R., Nagibin, V., Groth, C., Altevogt, P., . . . & Umansky, V. (2018). Targeting myeloid-derived suppressor cells to bypass tumor-induced immunosuppression. Frontiers in immunology, 9, 398.
32. Chinnasamy, D., Yu, Z., Theoret, M. R., Zhao, Y., Shrimali, R. K., Morgan, R. A., . . . & Rosenberg, S. A. (2010). Gene therapy using genetically modified lymphocytes targeting VEGFR-2 inhibits the growth of vascularized syngenic tumors in mice. The Journal of clinical investigation, 120(11), 3953-3968.

1. A method of treating a disease comprising administering to a subject a chimeric antigen receptor modified T cell (CAR T cell) and a STING agonist.

2. The method of claim 1, wherein the CAR T cell is a second generation CAR T cell or a third generation CAR T cell.

3. The method of claim 1, wherein the CAR T cell is a Th/Tc17 CAR T cell or a 7/15 CAR T cell.

4. (canceled)

5. The method of claim 1, wherein the STING agonist is a viral or bacterial product.

6. (canceled)

7. The method of claim 1, wherein the CAR T cell and STING agonist are administered concurrently.

8. The method of claim 1, wherein the CAR T cell and STING agonist are administered sequentially.

9. The method of claim 1, further comprising administering to the subject a myeloid derived suppressor cell (MDSC) depleting agent.

10. The method of claim 9, wherein the MDSC depleting agent is an antibody.

11. (canceled)

12. The method of claim 9, wherein the MDSC depleting agent is administered concurrently with the CAR T cell and the STING agonist.

13. The method of claim 9, wherein the MDSC depleting agent is administered sequentially with the CAR T cell and the STING agonist.

14. The method of claim 1, further comprising administering to the subject a PD-1 inhibitor.

15. The method of claim 14, wherein the PD-1 inhibitor is an anti-PD-1 antibody.

16. The method of claim 14, wherein the PD-1 inhibitor is administered concurrently with the CAR T cell and the STING agonist.

17. The method of claim 14, wherein the PD-1 inhibitor is administered sequentially with the CAR T cell and the STING agonist.

18. The method of claim 1, further comprising inactivating PD-1 in the CAR T cell using gene editing.

19. (canceled)

20. The method of claim 1, wherein the disease is cancer.

21. (canceled)

22. (canceled)

23. (canceled)

24. A method of treating cancer comprising administering to a subject a Th17 CAR T cell, a STING agonist, a MDSC depleting agent, and a PD-1 inhibitor.

25. The method of claim 24, wherein the Th17 CAR T cell, STING agonist, MDSC depleting agent, and the PD-1 inhibitor are administered concurrently.

26. The method of claim 24, wherein the Th17 CAR T cell, STING agonist, MDSC depleting agent, and the PD-1 inhibitor are administered sequentially.

27.-34. (canceled)

35. A method of producing Th/Tc17 CAR T cells comprising:
a. transducing CD4 and CD8 activated T cells with LH28z virus;
b. culturing the transduced T cells with Th17 cytokine cocktail;
c. reactivating the cultured T cells; and
d. culturing the reactivated T cells in Th17 cytokine cocktail plus IL-23.

36.-39. (canceled)
